(19) United States
(12) Patent Application Publication
(10) Pub. No.: US 2025/0188455 A1
(43) Pub. Date: Jun. 12, 2025

(54) LIPOPHILIC OLIGONUCLEOTIDE CONJUGATES

(71) Applicant: Vanderbilt University, Nashville, TN (US)

(72) Inventors: Craig L. Duvall, Nashville, TN (US); Ella Hoogenboezem, Nashville, TN (US)

(21) Appl. No.: 18/688,282

(22) PCT Filed: Sep. 2, 2022
(86) PCT No.: PCT/US2022/042445

§ 371 Date: Feb. 29, 2024

Related U.S. Application Data
(60) Provisional application No. 63/240,256, filed on Sep. 2, 2021.

(51) Int. Cl. C12N 15/113; A61K 47/54

(52) U.S. Cl. CPC C12N 15/113; A61K 47/543; A61K 47/554; C12N 2310/14; C12N 2310/315; C12N 2310/3515; C12N 2320/32

(57) ABSTRACT

Disclosed herein are lipophilic oligonucleotide conjugates that can bind to albumin. An example conjugate includes an oligonucleotide; a lipophilic ligand capable of binding albumin; and a linker attaching the oligonucleotide and the lipophilic ligand, the linker including a branching molecule attached to the oligonucleotide, and a hydrophilic spacer attaching the branching molecule and the lipophilic ligand. Also disclosed are compositions including the conjugate and methods of using the conjugate.

### CROSS-REFERENCE TO RELATED APPLICATIONS

This application is the U.S. national stage entry, under 35 U.S.C. § 371, of International Application Number PCT/US2022/042445, filed Sep. 2, 2022, which claims priority to U.S. Provisional Patent Application No. 63/240,256, filed on Sep. 2, 2021, the entire contents of which are incorporated herein by reference.

### STATEMENT REGARDING FEDERALLY SPONSORED RESEARCH

This invention was made with government support under grant numbers CA224241, EB019409, and CA260958, awarded by the National Institutes of Health. The government has certain rights in the invention.

### SEQUENCE LISTING

This sequence listing is filed with the application and is incorporated by reference herein. The sequence listing xml file “093386-9354-US02 Sequence Listing.xml” was created on Feb. 29, 2024, and is 24,835 bytes in size.

### TECHNICAL FIELD

The present disclosure relates to lipophilic oligonucleotide conjugates and their use in biomedical applications, such as drug delivery.

### INTRODUCTION

Short interfering RNA (siRNA) is a powerful gene silencing platform that has recently achieved clinical relevance with the approval of Alnylam Pharmaceuticals' Patisiran, Givosiran, and Lumasiran. The traditional approach for overcoming delivery barriers faced by siRNA, such as rapid renal clearance and nuclease degradation, is encapsulation in lipid- or polymer-based formulations. One such example is Patisiran, which packages siRNA inside of a lipid nanoparticle containing DLin-MC3-DMA and PEG₂₀₀₀-C-DMG. However, these lipid and polymer carrier formulations present challenges including their associated immune response. Accordingly, a delivery of siRNA without the use of lipids and/or polymer carriers (e.g., “carrier-free” delivery) may be beneficial.

### SUMMARY

In one aspect, disclosed are conjugates including an oligonucleotide; a lipophilic ligand capable of binding albumin; and a linker attaching the oligonucleotide and the lipophilic ligand, the linker comprising a branching molecule attached to the oligonucleotide, and a hydrophilic spacer attaching the branching molecule and the lipophilic ligand.

In another aspect, disclosed are conjugates including an siRNA; a lipophilic ligand capable of binding albumin, the lipophilic ligand including two independent lipids, each lipid including a C₁₈ hydrocarbon chain; and a linker attaching the siRNA and the lipophilic ligand, the linker including a branching molecule attached to the siRNA and including at least one branch point having at least two independent branches, and a hydrophilic spacer attaching an individual branch and an individual lipid, the hydrophilic spacer including 1 to 6 hydrophilic blocks, each hydrophilic block including 2 to 10 repeats of ethylene glycol.

In another aspect, disclosed are compositions including a disclosed conjugate and one or more pharmaceutically acceptable excipients.

In another aspect, disclosed are methods of gene silencing, the method including administering a disclosed conjugate to a subject in need thereof.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows example conjugates and points of analysis for structure-function relationships.

FIG. 2 shows that hydrophilic linker (also referred to herein as “hydrophilic spacer”) length can affect in vitro albumin-binding and micellar self-assembly of lipid-siRNA conjugates. (FIG. 2A) Native polyacrylamide gel electrophoresis (PAGE) gel of siRNA conjugates run in the presence or absence of human serum albumin. Albumin association is indicated by an upwards shift of nucleic acid staining to coincide with the molecular weight increase from albumin binding. (FIG. 2B) Representative in vitro binding responses of siRNA conjugates to human serum albumin (400 nM) measured by biolayer interferometry. Dashed line represents transition from association to dissociation. (FIG. 2C) Proposed mechanism of competition between self-assembly and albumin binding of siRNA conjugates. (FIG. 2D) Critical micelle concentration of siRNA conjugates observed after 2 hours of incubation with Nile Red at 37° C. (n=3). Significance assessed by one-way ANOVA with Tukey's multiple comparisons test.

FIG. 3 shows that hydrophilic linker length can influence in vivo pharmacokinetics, biodistribution, and in vivo albumin-binding of lipid-siRNA conjugates. (FIG. 3A) Representative intravital microscopy images of mouse ear vasculature at TOmin and T30 min. (FIG. 3B) Conjugate absolute half-lives calculated from intravital microscopy fluorescent traces in vasculature (n=3-4). Significance assessed by 1-way ANOVA with Tukey's multiple comparisons test. (FIG. 3C) Kidney biodistribution of fluorescently labeled conjugates approximately 45 minutes after 1 mg/kg intravenous injection (n=3). Significance assessed with 1-way ANOVA with Tukey's multiple comparison's test. (FIG. 3D) Representative fluorescent intensity traces of mouse plasma isolated approximately 45 minutes after 1 mg/kg intravenous injection separated into sequential fractions by size exclusion chromatography. (FIG. 3E) Fraction of conjugate bound to albumin in mouse plasma after 1 mg/kg intravenous injection as calculated from known standards and sum of fractions' fluorescent intensities (n=3). Significance assessed with 1-way ANOVA (Kruskal-Wallis) with Dunn's multiple comparisons test against control conjugate si-EG₄₅<L₂.

FIG. 4 shows that albumin-binding can increase tumor accumulation and carrier-free silencing of siRNA-lipid conjugates. (FIG. 4A) Tumor:kidney ratio of fluorescently labeled conjugate injected via tail vein 18 h before IVIS imaging (n=5-9). Significance assessed by 1-way ANOVA (Kruskal-Wallis) with Dunn's multiple comparisons test against siRNA control. (FIG. 4B) Luciferase activity of tumors normalized to saline treated controls measured ex vivo after 3×2.5 mg/kg injections via tail vein administration (n=5-9). Significance was assessed by 1-way ANOVA (Kruskal-Wallis) with Dunn's multiple comparisons test against PBS control. (FIG. 4C) Representative images of tumor immunohistochemistry using AlexaFluor488-labeled Anti-Firefly Luciferase antibody after 3×2.5 mg/kg injections via tail vein administration. (FIG. 4D) Quantification of relative MCL-1 mRNA levels after a single bolus injection of si_{MCL-1}<(EG₁₈L)₂ both 4 days and (FIG. 4E) 8 days after treatment. Saline group replotted with 8 d data but all significance tested together in a single 1-way ANOVA with Tukey's multiple comparison's test (n=4-6).

FIG. 5 shows that inclusion of a 5′ sense strand and linker phosphorothioate (PS) bonds can improve performance of lipid-modified siRNA conjugate in vitro and in vivo. (FIG. 5A) Structural PS variants of si<(EG₁₈L)₂ conjugate investigated, where red indicates phosphorothioate bonds and black phosphodiester. (FIG. 5B) Representative in vitro binding responses of PS variants to human serum albumin (400 nM) measured by biolayer interferometry. Dashed line represents transition from association to dissociation. (FIG. 5C) Critical micelle concentration observed after 2-hour incubation with Nile Red at 37° C. (n=3). Significance assessed by 1-way ANOVA with Tukey's multiple comparisons test. (FIG. 5D) Absolute circulation half-life measured by intravital microscopy (n=3-4). Significance assessed by 1-way ANOVA with Tukey's multiple comparisons test. (FIG. 5E) Organ biodistribution of conjugates measured by IVIS approximately 45 minutes after 1 mg/kg tail vein injection. Significance assessed by 2-way ANOVA with Tukey's multiple comparisons test. (FIG. 5F) Fraction of albumin-bound conjugate in plasma isolated from mice approximately 45 minutes after i.v. injection (n=3). Significance assessed by 1-way ANOVA with Tukey's multiple comparisons test.

FIG. 6 shows that proximal positioning of a divalent lipid-siRNA conjugate branching point can improve in vitro and in vivo outcomes. (FIG. 6A) Structures of branching position variants of conjugate investigated. (FIG. 6B) Representative in vitro binding responses of brancher variants to human serum albumin (400 nM) measured by biolayer interferometry. Dashed line represents transition from association to dissociation. (FIG. 6C) Critical micelle concentration observed after 2-hour incubation with Nile Red at 37° C. (n=3). Significance assessed by 1-way ANOVA with Tukey's multiple comparisons test. (FIG. 6D) Absolute circulation half-life measured by intravital microscopy (n=3-4). Significance assessed by 1-way ANOVA with Tukey's multiple comparisons test. (FIG. 6E) Organ biodistribution of conjugate measured by IVIS approximately 45 minutes after 1 mg/kg tail vein injection. Significance assessed by 2-way ANOVA with Tukey's multiple comparisons test. (FIG. 6F) Fraction of albumin-bound conjugate in plasma isolated from mice approximately 45 minutes after i.v. injection (n=3). Significance assessed by 1-way ANOVA with Tukey's multiple comparisons test.

FIG. 7 shows that carboxylic acid groups on termini of lipids increase albumin binding affinity but do not improve gene silencing, while including of an unstaturated bond in the lipid structures does not have any beneficial effect. (FIG. 7A) Absolute circulation half-life measured by intravital microscopy (n=3-5). Significance assessed by 1-way ANOVA with Tukey's multiple comparisons test. (FIG. 7B) Kidney biodistribution of fluorescently labeled conjugates approximately 45 minutes after 1 mg/kg intravenous injection (n=3-4). Significance assessed with 1-way ANOVA with Tukey's multiple comparison's test. (FIG. 7C) Fraction of albumin-bound conjugate in plasma isolated from mice approximately 45 minutes after i.v. injection (n=3-4). Significance assessed by 1-way ANOVA with Tukey's multiple comparisons test. (FIG. 7D) Representative in vitro binding responses of lipid variants to human serum albumin (400 nM) measured by biolayer interferometry. Dashed line represents transition from association to dissociation. (FIG. 7E) siRNA dosing regime in mice bearing orthotopic, luciferase-expressing MDA-MB-231 tumors (FIG. 7F) Epifluorescence of organs measured by IVIS approximately 18 h after injection with fluorescently labeled siRNA constructs. Significance assessed by 2-way ANOVA (n=5-6). (FIG. 7G) Average fluorescent intensity of tumor cells from each mouse measured by flow cytometry (n=5-6). Significance assessed by 1-way ANOVA with Tukey's multiple comparisons test. (FIG. 7H) Luciferase activity of tumors normalized to saline treated controls measured ex vivo after 3×1.5 mg/kg injections via tail vein administration (n=6). Significance was assessed by 1-way ANOVA with Tukey's multiple comparison's test.

FIG. 8 shows that reversibly bound siRNA conjugates can outperform siRNA duplex covalently bound to albumin. (FIG. 8A) Schematic depicting the design rationale of covalently bound siRNA duplex for comparison to reversibly bound siRNA conjugate. (FIG. 8B) Electrophoretic gel depicting the upward shift of the siRNA duplex band after conjugation to albumin. (FIG. 8C) Fraction of albumin-bound conjugate in plasma isolated from mice approximately 45 minutes after i.v. injection (n=3-4). Significance assessed by 1-way ANOVA with Tukey's multiple comparisons test. (FIG. 8D) Absolute circulation half-life measured by intravital microscopy (n=3-5). Significance assessed by 1-way ANOVA with Tukey's multiple comparisons test. (FIG. 8E) Kidney biodistribution of fluorescently labeled conjugates approximately 45 minutes after 1 mg/kg intravenous injection (n=3-4). Significance assessed with 1-way ANOVA with Tukey's multiple comparison's test. (FIG. 8F) siRNA dosing regime in mice bearing orthotopic, luciferase-expressing MDA-MB-231 tumors (FIG. 8G) Epifluorescence of organs measured by IVIS approximately 18 h after injection with fluorescently labeled siRNA constructs. Significance assessed by 2-way ANOVA (n=5-6). (FIG. 8H) Average fluorescent intensity of tumor cells from each mouse measured by flow cytometry (n=5-6). Significance assessed by 1-way ANOVA with Tukey's multiple comparisons test. (FIG. 8I) Luciferase activity of tumors normalized to saline treated controls measured ex vivo after 3×1.5 mg/kg injections via tail vein administration (n=6). Significance was assessed by 1-way ANOVA with Tukey's multiple comparison's test.

FIG. 9 shows siRNA modification pattern validation and stability. (FIG. 9A) Graphical schematic of structural differences between synthetic Dicer Substrate siRNA and alternating 2′F-, 2′OMe-modified “Zipper” siRNA. (FIG. 9B—left panel) Lipofection-mediated knockdown of 25 nM siRNA in Luciferase-expressing MDA-MB-231 is. Luminescent signal was normalized to scrambled control siRNAs to account for any nonspecific toxicity effects. (FIG. 9B—right panel) Serum stability of siRNA challenged with 60% fetal bovine serum at 37° C. visualized by agarose gel electrophoresis.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th}Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “alkenyl” refers to a straight or branched, unsaturated hydrocarbon chain containing at least one carbon-carbon double bond and from 2 to 10 carbon atoms.

The term “alkyl” refers to a straight or branched, saturated hydrocarbon chain containing from 1 to 10 carbon atoms. The term “C₁-C₃ alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkynyl” refers to a type of alkyl group in which the first two atoms of the alkyl group form a triple bond. That is, an alkynyl group begins with the atoms —C═C—R, wherein R refers to the remaining portions of the alkynyl group, which may be the same or different. Non-limiting examples of an alkynyl group include —C═CH, —C═CH₃ and —C═CCH₂CH₃. The “R” portion of the alkynyl moiety may be branched, straight chain, or cyclic.

The term “amino,” as used herein, refers to —NH₂.

The term “attached” refers to two moieties being attached through a bond where there can be intervening moieties or molecules in between. For example, the branching molecule can be attached to the oligonucleotide by having an intervening moiety, such as a second linker, between the oligonucleotide and the linker. Attached can also include “directly attached,” which refers to two moieties being attached through a bond with no other intervening moieties or molecules.

The term “carboxyl” refers to the group —C(═O)OR, wherein R is selected from the group consisting of hydrogen, alkyl, alkenyl, and alkynyl, any of which may be optionally substituted, e.g., with one or more substituents.

The term “effective amount” or “therapeutically effective amount” refers to an amount sufficient to effect beneficial or desirable biological and/or clinical results.

The term “hydroxyl” refers to an —OH group.

The term “oligonucleotide” refers to a polymer of nucleotides. The terms “polynucleotide,” “nucleic acid,” and “oligonucleotide”, may be used interchangeably herein. Typically, a polynucleotide comprises at least three nucleotides. Oligonucleotides can be single stranded or double stranded. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The oligonucleotide can have a modified backbone, such as having the sugar phosphate backbone being replaced with a peptide or peptide-like backbone (e.g., peptide nucleic acids). The oligonucleotide can also include modified bases that contain a methylene bridge bond between the 2′ oxygen and the 4′ carbon of the pentose ring (e.g., locked nucleic acids). Example oligonucleotides include, but are not limited to, DNA and RNA, such as RNAi, siRNA, or shRNA.

The terms “subject” or “subject in need thereof” refer to a target of administration, which optionally displays symptoms related to a particular disease, pathological condition, disorder, or the like. The subject of the disclosed methods can be a vertebrate, such as a mammal, a fish, a bird, a reptile, or an amphibian. Thus, the subject of the disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The terms “treatment” or “treating” refer to the medical management of a subject with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### 2. Conjugates

Provided herein are lipophilic oligonucleotide conjugates that can overcome limitations of previous oligonucleotide formulations by advantageously binding to albumin. The conjugate includes an oligonucleotide, a lipophilic ligand capable of binding albumin, and a linker attaching the oligonucleotide and the lipophilic ligand. The linker includes a branching molecule attached to the oligonucleotide and a hydrophilic spacer attaching the branching molecule to the lipophilic ligand.

The oligonucleotide, the linker, and the lipophilic ligand can be attached to each other through various types of linkages/bonds. For example, the oligonucleotide, the linker, and/or the lipophilic ligand can be attached through phosphorothioate bonds, phosphodiester bonds, a cleavable linker (e.g., deoxythymidine (dT), pH-cleavable bond such as ketal), or a combination thereof. In some embodiments, the oligonucleotide, the linker, and/or the lipophilic ligand are attached through phosphorothioate bonds. In some embodiments, the conjugate includes about 20% to about 60% phosphorothioate linkages based on the total amount of phosphate-based linkages in the conjugate, such as about 20% to about 55% phosphorothioate linkages, about 25% to about 50% phosphorothioate linkages, about 35% to about 45% phosphorothioate linkages, or about 40% to about 45% phosphorothioate linkages—based on the total amount of phosphate-based linkages in the conjugate. The combination of phosphodiester linkages and phosphorothioate linkages can be referred to as the total amount of phosphate-based linkages, and is not inclusive of potential phosphorylation of sequences, e.g., to avoid deactivation of phosphatases.

The conjugate's arrangement and composition can provide advantageous benefits, such as being able to bind albumin while also minimizing its propensity to self-assemble into micelles. For example, the conjugate can have a binding affinity (K_{d}) to albumin of less than 1 μM, less than 500 nM, less than 250 nM, less than 100 nM, less than 80 nM, less than 60 nM, less than 50 nM, less than 45 nM, less than 40 nM, or less than 35 nM. In some embodiments, the conjugate has a K_{d} to albumin of greater than 0.1 nM, greater than 0.2 nM, greater than 0.4 nM, greater than 0.5 nM, greater than 0.6 nM, greater than 0.7 nM, greater than 0.8 nM, greater than 0.9 nM, greater than 1 nM, or greater than 5 nM. In some embodiments, the conjugate has a K_{d} to albumin of about 0.1 nM to about 1 μM, such as about 0.5 nM to about 500 nM, about 0.8 nM to about 100 nM, about 0.5 nM to about 100 nM, about 1 nM to about 50 nM, or about 5 nM to about 35 nM. The conjugate can reversibly bind albumin. In some embodiments, the conjugate does not covalently bind to albumin.

In addition, the conjugate can have a critical micelle concentration of greater than 1850 nM, greater than 1900 nM, greater than 1950 nM, greater than 2000 nM, greater than 2100 nM, greater than 2200 nM, greater than 2300 nM, greater than 2400 nM, greater than 2500 nM, greater than 3000 nM, or greater than 3500 nM. In some embodiments, the conjugate has a critical micelle concentration of less than 4500 nM, less than 4000 nM, less than 3500 nM, less than 3000 nM, less than 2500 nM, less than 2400 nM, less than 2300 nM, less than 2200 nM, less than 2100 nM, less than 2000 nM, or less than 1950 nM. In some embodiments, the conjugate has a critical micelle concentration of about 1850 nM to about 4000 nM, such as about 1900 nM to about 3500 nM, about 2000 nM to about 3500 nM, about 1850 nM to about 3500 nM, or about 2500 nM to about 3500 nM.

As discussed elsewhere, the conjugate can bind albumin through the lipophilic ligand. In some embodiments, the binding of the conjugate to the serum protein albumin enhances the pharmacokinetic properties of the oligonucleotide as compared to an unmodified oligonucleotide and/or existing nanocarrier including the oligonucleotide. In some embodiments, enhancing the pharmacokinetic properties includes increasing the circulation half-life and/or bioavailability of the oligonucleotide, as compared to an unmodified oligonucleotide and/or existing nanocarrier including the oligonucleotide. Additionally, enhancing the pharmacokinetic properties may include increasing the quantity of cellular or tumor accumulation, increasing the homogeneity of cellular or tumor accumulation, increasing resistance to nucleases, and/or permitting increased dosing amount with decreased toxicity as compared to an unmodified oligonucleotide and/or existing nanocarrier including the oligonucleotide.

In some embodiments, the conjugate includes an siRNA; a lipophilic ligand capable of binding albumin, the lipophilic ligand comprising two independent lipids, each lipid including a C18 hydrocarbon chain; and a linker attaching the siRNA and the lipophilic ligand, the linker comprising a branching molecule attached to the siRNA and including at least one branch point having at least two independent branches, and a hydrophilic spacer attaching an individual branch and an individual lipid, the hydrophilic spacer including 1 to 6 hydrophilic blocks, each hydrophilic block including 2 to 10 repeats of ethylene glycol.

### A. Oligonucleotide

The conjugate includes an oligonucleotide. The oligonucleotide can instill a therapeutic and/or beneficial property to the conjugate. The oligonucleotide can be single stranded or double stranded. An example of a single stranded oligonucleotide includes, but is not limited to, single stranded antisense oligonucleotides (e.g., single stranded antisense DNA and/or RNA). The oligonucleotide can include DNA, RNA, a synthetic mimic of DNA or RNA, or a combination thereof. In some embodiments, the oligonucleotide includes DNA, RNA, or a synthetic mimic of DNA or RNA. In some embodiments, the oligonucleotide includes RNA. In some embodiments, the oligonucleotide is RNA. Example RNAs include, but are not limited to, siRNA, miRNA, and an antisense RNA. In some embodiments, the oligonucleotide includes siRNA, miRNA or single stranded antisense RNA.

The benefits realized from the end modification of the disclosed conjugate (e.g., attaching the lipophilic ligand to the oligonucleotide through the linker) can be used with any desirable oligonucleotide. In other words, the oligonucleotide of the conjugate is sequence agnostic. This modification can be added to either single or double stranded oligonucleotides that contain either natural or modified nucleotide bases. The single or double stranded nucleotides can also be of variable length, such as 10 to 30 bases in length.

For an example process of selecting an oligonucleotide, such as siRNA, the sequence can be first determined using publicly available prediction algorithms. These algorithms can generate many candidate sequences for targeting any given gene. These potential sequences are first screened for on-target gene silencing potency in vitro (without chemical modifications). After identification of one or more potent sequences, chemical modifications can be added to the sequence, and it can be rescreened for in vitro gene silencing activity prior to screening for albumin binding affinity and pharmacokinetic/pharmacodynamic behaviors in vivo. The disclosed albumin binding end chemistry may be successfully integrated with multiple sequences targeting any single gene and may also be adapted for delivery of sequences against theoretically any gene of interest.

The oligonucleotide can include a plurality of stabilizing modifications. Examples of stabilizing modifications include, but are not limited to, phosphorothioate linkages, 2′F modification, 2′OMe modification, and combinations of 2′F and 2′OMe modifications (e.g., zipper pattern). In addition, the oligonucleotide can include both phosphodiester linkages and phosphorothioate linkages. In some embodiments, the oligonucleotide includes a plurality of phosphorothioate linkages. In some embodiments, the oligonucleotide includes phosphorothioate linkages at its terminal end(s). In some embodiments, the oligonucleotide includes about 1% to about 30% phosphorothioate linkages based on a total amount of phosphate-based linkages in the oligonucleotide, such as about 10% to about 25% phosphorothioate linkages or about 15% to about 24% phosphorothioate linkages—based on a total amount of phosphate-based linkages in the oligonucleotide. In some embodiments, the oligonucleotide includes about 21% phosphorothioate linkages based on a total amount of phosphate-based linkages in the oligonucleotide.

The oligonucleotide can have a varying amount of nucleotides. For example, the oligonucleotide can have about 15 to about 40 nucleotides, such as about 16 to about 38 nucleotides, about 15 to about 35 nucleotides, about 18 to about 32 nucleotides, about 18 to about 30 nucleotides, or about 20 to about 35 nucleotides.

In some embodiments, the oligonucleotide includes a nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or a combination thereof. In some embodiments, the oligonucleotide includes a nucleotide sequence of SEQ ID NO:7, SEQ ID NO:8, or a combination thereof.

### B. Lipophilic Ligand

The lipophilic ligand is capable of binding albumin, and thus can instill in the conjugate the ability to bind albumin. The lipophilic ligand can include any lipophilic moiety suitable for binding into a fatty acid pocket of albumin. In some embodiments, the lipophilic ligand includes a lipid with a long hydrocarbon chain.

The lipid can include a C₁₂-C₂₂ hydrocarbon chain, such as a C₁₂-C₂₀ hydrocarbon chain, a C₁₄-C₂₂ hydrocarbon chain, a C₁₆-C₂₂ hydrocarbon chain, or a C₁₆-C₂₀ hydrocarbon chain. In some embodiments, the lipid includes a C₁₈ hydrocarbon chain. The lipid can be saturated or unsaturated. In addition, the lipid may have a terminal end. The terminal end may include a functional group that may aid in binding. In some embodiments, the terminal end of the lipid includes an alkyl, carboxyl, hydroxyl, or amino. In some embodiments, the terminal end of the lipid includes an alkyl or carboxyl. In embodiments where there is more than one lipid, each lipid can include a different functional group at its terminal end. For example, one terminal end can include an alkyl and one terminal end can include a carboxyl. In some embodiments, the terminal end includes an alkyl. In some embodiments, the terminal end of the lipid does not include a hydroxyl or a carboxyl.

The lipophilic ligand can include more than one lipid. Having more than one lipid can allow for multivalency of the lipophilic ligand and the conjugate thereof. The lipophilic ligand can include at least 2 lipids, at least 3 lipids, at least 4 lipids, at least 5 lipids, at least 6 lipids, at least 7 lipids, or at least 8 lipids. In some embodiments, the lipophilic ligand includes less than 10 lipids, less than 9 lipids, less than 8 lipids, less than 7 lipids, less than 6 lipids, or less than 5 lipids. In some embodiments, the lipophilic ligand includes 1 to 10 lipids, such as 1 to 8 lipids, 1 to 6 lipids, 2 to 8 lipids, 2 to 6 lipids, 2 to 4 lipids, or 2 to 3 lipids.

The lipophilic ligand can be divalent. For example, the lipophilic ligand can include two independent lipids. In some embodiments, the lipophilic ligand incudes two independent lipids, each lipid including a C₁₂-C₂₂ hydrocarbon chain. In some embodiments, the lipophilic ligand includes two independent lipids, each lipid including a C₁₈ hydrocarbon chain.

The lipophilic ligand and lipid(s) can be attached to the hydrophilic spacer. In some embodiments, the lipophilic ligand and lipid(s) are directly attached to the hydrophilic spacer. In some embodiments, the lipophilic ligand includes two individual lipids, each lipid bound to a separate, individual hydrophilic spacer which is bound to a separate branch of the branching molecule. In such embodiments, the lipids may be the same or different. For example, the lipids can both include C₁₈ hydrocarbon chains. Alternatively, in other embodiments, the lipids can include hydrocarbon chains of varying length. In some embodiments, the lipophilic ligand includes two distinct types of lipids.

### C. Linker

The lipophilic ligand is attached to the oligonucleotide through a linker. The arrangement and composition of the linker can provide the conjugate with advantageous properties, such as, but not limited to, binding to albumin, decreased propensity to self-assembly into micelles, and improved pharmacokinetics. The linker includes a branching molecule and a hydrophilic spacer. The linker can further include other types of spacers and/or linkers known within the art.

i. Branching Molecule

The branching molecule can be any suitable molecule that allows for branching of the conjugate, e.g., extending from the oligonucleotide. Example branching molecules include, but are not limited to, a phosphoramidite (e.g., symmetrical branching CED phosphoramidite), a tri-valent splitter, or a tetra-valent splitter.

The branching molecule can be positioned between the oligonucleotide and the hydrophilic spacer. Or in other words, the branching molecule can be attached to the oligonucleotide and the hydrophilic spacer. The branching molecule can also be directly attached to the oligonucleotide. In some embodiments, the branching molecule is directly attached to the oligonucleotide and attached to the hydrophilic spacer. In some embodiments, the branching molecule is directly attached to the oligonucleotide and directly attached to the hydrophilic spacer. The branching molecule can be directly attached, or conjugated, to the oligonucleotide through a phosphorothioate bond, phosphodiester, or cleavable linker (e.g., deoxythymidine (dT), pH-cleavable bond such as ketal). Although, in some embodiments, there are no intervening moieties or molecules when directly attached, as will be appreciated by those skilled in the art, the placement of the branch point within the branching molecule may be adjusted based upon the structure of the branching molecule itself.

The branching molecule can have multiple, independent branch points, each branch point having at least two independent branches. For example, the branching molecule can have at least 2 branch points, at least 3 branch points, at least 4 branch points, or at least 5 branch points. In some embodiments, the branching molecule has less than 7 branch points, less than 6 branch points, less than 5 branch points, or less than 4 branch points. In some embodiments, the branching molecule has 1 to 5 branch points, such as 1 to 4 branch points, 1 to 3 branch points, or 1 to 2 branch points.

Each branch point can have multiple, independent branches. For example, each branch point can have at least 2 branches, at least 3 branches, at least 4 branches, at least 5 branches, at least 6 branches, at least 7 branches, at least 8 branches, at least 9 branches, or at least 10 branches. In some embodiments, each branching point can have less than 12 branches, less than 11 branches, less than 10 branches, less than 9 branches, less than 8 branches, less than 7 branches, less than 6 branches, less than 5 branches, or less than 4 branches. In some embodiments, each branch point has 2 to 12 branches, such as 2 to 10 branches, 2 to 8 branches, 2 to 6 branches, or 2 to 4 branches. In some embodiments, each branch point has 2 branches.

The branching molecule's positioning in the conjugate can play an important role in determining properties of the overall conjugate. For example, attaching the branching molecule to the oligonucleotide, rather than to the lipophilic ligand, and having the hydrophilic spacer between the branching molecule and the lipophilic ligand unexpectedly provides improved properties. It is hypothesized, without wishing to be bound by a particular theory, that the positioning of the hydrophilic spacer after the branching molecule can provide additional flexibility and separation between the lipophilic ligand and lipid(s) thereof. The lipophilic ligand and lipid(s) thereof with this additional flexibility and separation show higher affinity for albumin. Additionally, and again without wishing to be bound by a particular theory, it is hypothesized that attaching the branching molecule to the oligonucleotide can decrease the self-micellization of the conjugate, which in turn can allow the conjugate to remain more unimeric in solution, and thus can be more available for binding to albumin. In addition, this may aid in binding to the outer surface of a cell membrane, which can promote internalization. In contrast, when the hydrophilic spacer is positioned before the branching molecule the lipophilic ligand and lipid(s) thereof can be more closely spaced, which can cause the conjugate to self-assemble, e.g., into a micelle, thereby limiting the lipophilic ligand's ability to interact with albumin.

The branching molecule can be included in the conjugate as a way to introduce multifunctionality, such as multivalency, to the conjugate. For example, the branching molecule can be used to increase the valency of the lipophilic ligand and the conjugate. The branching molecule can have independent branches that are attached to independent lipids. In some embodiments, the branching molecule has 2 independent branches that are attached to 2 independent lipids. The same can be said if there are 3 independent branches, these individual branches can be attached to 3 independent lipids. However, in some embodiments, not every branch is attached to a lipid. For example, in some embodiments, the branching molecule can have 4 branches, where only 2 of the 4 branches are attached to a lipid. Varying combinations of branches and their attachment to lipids can be used for the disclosed conjugate.

ii. Hydrophilic Spacer

The hydrophilic spacer can include any suitable hydrophilic compound for attaching the lipophilic ligand to the branching molecule. Examples of suitable hydrophilic compounds include, but are not limited to, ethylene glycol, zwitterionic linkers, peptoids (e.g., poly(sarcosine)), amino acids, poly(ethylene glycol) substitutes including: poly(glycerols), poly(oxazoline), poly(acrylamide), poly (N-acryloyl morpholine, poly(N,N-dimethyl acrylamide), poly(2-hydroxypropyl methacrylamide), poly(2-hydroxyethyl methacryalmide), and any other similar hydrophilic spacer molecule and/or polymer.

The hydrophilic spacer can be attached to the lipophilic ligand and the branching molecule. As mentioned above, the branching molecule can include a branching point having at least two independent branches. Each branch of the branching molecule can be attached to an individual hydrophilic spacer. In addition, each hydrophilic spacer can be individually attached to an individual lipid of the lipophilic ligand. In some embodiments, the hydrophilic spacer is attached to the lipophilic ligand, the branching molecule, or both through phosphorothioate bonds. In some embodiments, the hydrophilic spacer is attached to a lipid of the lipophilic ligand, the branching molecule, or both through phosphorothioate bonds.

The hydrophilic spacer can include at least one hydrophilic block. For example, the hydrophilic spacer can include 1 to 100 hydrophilic blocks, such as 1 to 50 hydrophilic blocks, 1 to 20 hydrophilic blocks, as 1 to 18 hydrophilic blocks, 2 to 15 hydrophilic blocks, 3 to 10 hydrophilic blocks, 2 to 10 hydrophilic blocks, 1 to 15 hydrophilic blocks, 1 to 10 hydrophilic blocks, 2 to 8 hydrophilic blocks, 2 to 6 hydrophilic blocks, or 1 to 7 hydrophilic blocks. In some embodiments, the hydrophilic spacer includes 5 hydrophilic blocks. The hydrophilic blocks can be attached to each other, the branching molecule, and/or the lipophilic ligand. For example, the hydrophilic blocks can be attached to each other, the branching molecule, and/or the lipophilic ligand through phosphorothioate bonds, phosphodiester, or a cleavable linker (e.g., deoxythymidine (dT), pH-cleavable bond such as ketal). In some embodiments, each of the hydrophilic blocks are attached to each other through phosphorothioate linkages.

The hydrophilic block can include repeats of the hydrophilic compound. In some embodiments, the hydrophilic spacer includes 1 to 100 hydrophilic blocks (as described above), with each of the repeating blocks including 1 to 150 repeats of the hydrophilic compound, such as 1 to 100 repeats of the hydrophilic compound, 2 to 50 repeats of the hydrophilic compound, 1 to 45 repeats of the hydrophilic compound, 1 to 30 repeats of the hydrophilic compound, 2 to 20 repeats of the hydrophilic compound, or 2 to 10 repeats of the hydrophilic compound. In some embodiments, each hydrophilic block includes less than 150 repeats of the hydrophilic compound, less than 100 repeats of the hydrophilic compound, less than 75 repeats of the hydrophilic compound, less than 50 repeats of the hydrophilic compound, less than 45 repeats of the hydrophilic compound, less than 40 repeats of the hydrophilic compound, or less than 35 repeats of the hydrophilic compound. In some embodiments, each hydrophilic block includes greater than 2 repeats of the hydrophilic compound, greater than 3 repeats of the hydrophilic compound, greater than 4 repeats of the hydrophilic compound, greater than 5 repeats of the hydrophilic compound, greater than 6 repeats of the hydrophilic compound, greater than 7 repeats of the hydrophilic compound, or greater than 8 repeats of the hydrophilic compound.

In some embodiments, the hydrophilic spacer includes 1 to 10 hydrophilic blocks, with each block including 1 to 15 repeats of the hydrophilic compound. In some embodiments, the hydrophilic spacer includes 1 to 10 hydrophilic blocks, with each block including 1 to 10 repeats of the hydrophilic compound. In some embodiments, the hydrophilic spacer includes 1 to 6 hydrophilic blocks, with each block including 2 to 10 repeats of the hydrophilic compound. In some embodiments, the hydrophilic spacer includes 2 to 6 hydrophilic blocks, with each block including 3 to 8 repeats of the hydrophilic compound.

The hydrophilic compound can be included in different variations as part of the hydrophilic block. For example, the hydrophilic spacer can include 1 block including 150 repeats of the hydrophilic compound, 2 blocks each including 50 repeats of the hydrophilic compound, 5 blocks each including 6 repeats of the hydrophilic compound, 2 blocks—one block including 5 repeats of the hydrophilic compound and the other block including 10 repeats of the hydrophilic compound, or any combination of blocks and repeats as disclosed herein.

In some embodiments, the hydrophilic spacer includes a plurality of ethylene glycol repeats. For example, the hydrophilic spacer can include 1 to 150 ethylene glycol repeats, 1 to 120 ethylene glycol repeats, 1 to 100 ethylene glycol repeats, 1 to 90 ethylene glycol repeats, 1 to 80 ethylene glycol repeats, 1 to 70 ethylene glycol repeats, 1 to 60 ethylene glycol repeats, 1 to 50 ethylene glycol repeats, 1 to 40 ethylene glycol repeats, 1 to 30 ethylene glycol repeats, 2 to 150 ethylene glycol repeats, 3 to 150 ethylene glycol repeats, 4 to 150 ethylene glycol repeats, 5 to 150 ethylene glycol repeats, 6 to 150 ethylene glycol repeats, 7 to 150 ethylene glycol repeats, 8 to 150 ethylene glycol repeats, 9 to 150 ethylene glycol repeats, 10 to 150 ethylene glycol repeats, 10 to 140 ethylene glycol repeats, 10 to 130 ethylene glycol repeats, 10 to 120 ethylene glycol repeats, 10 to 110 ethylene glycol repeats, 10 to 100 ethylene glycol repeats, 10 to 90 ethylene glycol repeats, 10 to 80 ethylene glycol repeats, 10 to 70 ethylene glycol repeats, 10 to 60 ethylene glycol repeats, 6 to 60 ethylene glycol repeats, 6 to 40 ethylene glycol repeats, 8 to 35 ethylene glycol repeats, or 10 to 32 ethylene glycol repeats. The ethylene glycol repeats can be included as a hydrophilic block in different variations as described above.

In some embodiments, the hydrophilic spacer includes 1 and 10 hexaethylene glycol blocks (e.g., blocks of six ethylene glycol repeats). In some embodiments, the hydrophilic spacer includes 1 and 5 hexaethylene glycol blocks. In some embodiments, the hexaethylene glycol blocks are directly attached to each other and/or the branching molecule. In some embodiments, the hexaethylene glycol blocks are attached to each other and/or the branching molecule through phosphorothioate bonds, phosphodiester, or cleavable linker (e.g., deoxythymidine (dT), pH-cleavable bond such as ketal).

In some embodiments, the length of the hydrophilic spacer may be adjusted to provide desired properties. For example, ethylene glycol spacers with 18 ethylene glycol repeats can provide a higher binding to albumin. Alternatively, shorter spacers can yield conjugates with increased hydrophobicity, which can result in more tendency to bind lipoprotein complexes in the blood.

### D. Synthesis of Conjugates

Also provided herein are methods of synthesizing the conjugates. In some embodiments, the method includes solid phase synthesis where the full molecule is made/grown from a solid support, as opposed to solution phase conjugation of the oligonucleotide to the linkers/lipidic moieties post-solid phase synthesis. For example, the branching molecule can be integrated during the solid phase synthesis. The integration of the branching molecule can convert the linear growth to divalent growth (or any number of valency), where the hydrophilic spacers can be added to the two growing chains following the branch point.

### 3. Uses of the Conjugates

### A. Compositions

Also disclosed herein are compositions that include the conjugate and one or more pharmaceutically acceptable excipients. Examples of pharmaceutically acceptable excipients include, but are not limited to, buffering agents (e.g., phosphate buffered saline, artificial cerebrospinal fluid (aCSF), etc.), carbohydrates (e.g., glucose, trehalose, starch, etc.) solubilizers, solvents, antimicrobial preservatives, antioxidants, suspension agents, or a combination thereof. In some embodiments, the composition does not include a carrier composition, such as a polymer- or lipid-based formulation. The description of the conjugate, lipophilic ligand, oligonucleotide, linker, branching molecule, and hydrophilic spacer above may be applied to the disclosed compositions.

i. Administration

The composition can be administered prophylactically or therapeutically. In prophylactic administration, the composition can be administered in an amount sufficient to induce a response. In therapeutic applications, the composition can be administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The composition may be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Compositions can be administered using a regimen which maintains plasma levels above the MEC for 10-90% of the time, such as between 30-90% or between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

### A. Methods of Gene Silencing

Further provided herein are methods of gene silencing using the conjugate or composition thereof to a subject in need thereof. Albumin is the most abundant protein in human serum and is naturally rescued from renal or tissue clearance because it is recycled by the FcRn receptor, leading to recycling back into the blood stream. Due to its extended half-life in the body of approximately 19 days, its natural ability to transport fatty acids and other hydrophobic cargo, and its accumulation at sites of inflammation and vascular leakiness, albumin is an appealing drug carrier. Notably, albumin is also preferentially taken up and used as an amino acid source by cancer cells to meet their high energy needs. Accordingly, in some embodiments, the improved albumin binding of the conjugates disclosed herein provides improved drug delivery and/or uptake by cancer cells.

While oligonucleotide-based therapeutics can be limited by rapid renal clearance, nuclease degradation, and inability to target/penetrate cells of interest, the conjugates disclosed herein can provide improved circulation half-life, can shield the oligonucleotide from nucleases, and/or can provide extrahepatic delivery of the oligonucleotides. In addition, these advantages can be done without an associated carrier composition, such as a polymer or lipid formulation. Accordingly, in some embodiments the conjugate or composition thereof is administered without an associated carrier composition.

Given that albumin is a serum protein, the conjugate or composition thereof can be administered intravenously. Following administration, the conjugate can bind albumin. In some embodiments, the conjugate is pre-complexed with albumin prior to administration.

The method can be used to treat cancer. For example, cells in nutrient starved microenvironments characteristic of tumors can preferentially internalize albumin, thus can provide a mechanism for cell entry and targeted delivery thereto. In some embodiments, the method includes delivering an oligonucleotide to an extrahepatic target by administering one or more of the conjugates disclosed herein to a subject in need thereof. In some embodiments, the method includes treating cancer by administering one or more of the conjugates disclosed herein to a subject in need thereof. Example cancers include, but are not limited to, solid tumor malignancies, such as breast, pancreas, hepatic, bile duct (cholangiocarcinoma), and lung.

The description of the conjugate, composition, lipophilic ligand, oligonucleotide, linker, branching molecule, and hydrophilic spacer above may be applied to the disclosed methods.

The disclosed invention has multiple aspects, illustrated by the following non-limiting examples.

### 4. EXAMPLES

### Example 1

### Materials & Methods

Reagents. 2′-O-Me and 2′-F phosphoramidites, universal synthesis columns (MM1-2500-1), and all ancillary RNA synthesis reagents were purchased from Bioautomation. Symmetrical branching CED phosphoramidite was obtained from ChemGenes (CLP-5215). Cyanine 5 phosphoramidite (10-5915), stearyl phosphoramidite (10-1979), biotin TEG phosphoramidite (10-1955), hexaethyleneglycol phosphoramidite (10-1918), TEG cholesterol phosphoramidite (10-1976), 5′-Amino-Modifier 5 (10-1905), and desalting columns (60-5010) were all purchased from Glen Research. All other reagents were purchased from Sigma-Aldrich unless otherwise specified.

Conjugate Synthesis, Purification, and Validation. Oligonucleotides were synthesized using modified (2′-F and 2′-O-Me) phosphoramidites with standard protecting groups on a MerMade 12 Oligonucleotide Synthesizer (Bioautomation). Amidites were dissolved at 0.1M in anhydrous acetonitrile with the exception of 2′OMe U-CE phosphoramidite, which utilized 20% anhydrous dimethylformamide by volume as a cosolvent, and stearyl phosphoramidite, which was dissolved in 3:1 dichloromethane:acetonitrile by volume. Coupling was performed under standard conditions, and strands were grown on controlled pore glass with a universal terminus (1 μmol scale, 1000 Å pore size.)

Strands were cleaved and deprotected using 1:1 methylamine:40% ammonium hydroxide at room temperature for 2 hours. Lipophilic RNAs were purified by reversed-phase high performance liquid chromatography using a Clarity Oligo-RP column (Phenomenex) under a linear gradient from 85% mobile phase A (50 mM triethylammonium acetate in water) to 100% mobile phase B (methanol) or 95% mobile phase A to 100% mobile phase B (acetonitrile). Oligonucleotide containing fractions were then dried using a Savant SpeedVac SPD 120 Vacuum Concentrator (ThermoFisher). Conjugates were then resuspended in nuclease free water and sterile filtered before lyophilization.

Conjugate molecular weight and purity was confirmed using Liquid Chromatography-Mass Spectrometry (LC-MS) analysis on a ThermoFisher LTQ Orbitrap XL Linear Ion Trap Mass Spectrometer. Chromatography was performed using a Waters XBridge Oligonucleotide BEH C₁₈ Column under a linear gradient from 85% A (16.3 mM triethylamine—400 mM hexafluoroisopropanol) to 100% B (methanol) at 45° C. Control conjugate, si-EG₄₅L₂, molecular weight was validated using MALDI-TOF mass spectrometry using 50 mg/mL 3-hydroxypicolinic acid in 50% water, 50% acetonitrile with 5 mg/mL ammonium citrate as a matrix.

Synthesis of amine-reactive lipids and subsequent modification of oligonucleotides was adapted from methods reported by Prakash, T. P. et al. Fatty acid conjugation enhances potency of antisense oligonucleotides in muscle. Nucleic Acids Res 47, 6029-6044 (2019), which is incorporated herein by reference in its entirety. Briefly, amine-terminated oligonucleotides were speed vacuumed to dryness and desalted to remove MMT groups. Oligonucleotides were then lyophilized followed by reconstitution in 0.1 sodium tetraborate (pH 8.5) to a concentration of 500 uM. PFP-modified lipid was dissolved into a mixture of acetonitrile, DMSO, and triethylamine (70:29:1 by volume) at a concentration of 7 uM. Aqueous oligonucleotide was added dropwise to the organic solution for a 1:40 molar ratio of oligonucleotide-amine:amine-reactive lipid (approximately 25% 0.1M sodium tetraborate, 75% organic mixture). Solution was stirred overnight and desalted prior to purification and characterization detailed in main methods section.

Purified oligonucleotide was resuspended in 0.9% sterile saline and annealed to its complementary strand by heating to 95° C. and cooling stepwise by 15° C. every 9 min until 25° C. on a T100 Thermal Cycler (BioRad).

Duplexes directly bound to albumin were synthesized in a two-step, one-pot reaction. Briefly, conjugate covalently bound to albumin was synthesized by first reacting azido-PEG₃-maleimide (Click Chemistry Tools) with the free thiols on human (1 free SH) or mouse (2 free SH). Albumin was dissolved in PBS with 0.5M EDTA to a final concentration of 10 mM. Anhydrous DMF was used to solubilize and activate azido-PEG₃-maleimide. DBCO-modified siRNA duplex in PBS was reacted at a 1:1 ratio of DBCO groups:free SH groups and allowed to incubate at room temperature for 4 hours. To remove any siRNA that did not react with albumin, or reacted only with the azido linker, the resulting solution underwent 10 rounds of centrifugation in a 30 kDa cutoff Amicon filter at 14,000×g for 10 minutes for each round. Conjugation was confirmed by gel electrophoresis of precusor DBCO-siRNA alongside resulting siRNA-DBCO-albumin.

Cell Culture. Cells were cultured in Dulbecco's modified eagle's medium (DMEM, Gibco), containing 4.5 g/L glucose, 10% FBS (Gibco), and 50 μg/mL gentamicin. All cells were tested for Mycoplasma contamination MycoAlert Myocplasma Detection Kit (Lonza).

In Vitro Knockdown Experiments. For lipofection-mediated knockdown experiments, luciferase-expressing MDA-MB-231s were seeded at 4,000 cells per well in 96 well plates in complete media. After 24 h, cells were treated with siRNA (25 nM) using Lipofectamine 2000 (ThermoFisher) in OptiMEM according to manufacturer protocol, replacing with complete media at 24 h post-transfection, and measuring luciferase activity at 48 h post-transfection in cells treated for 5 min with 150 μg/mL D-Luciferin, potassium salt (ThermoFisher) using an IVIS Lumina III imaging system (Caliper Life Sciences).

Serum Stability. siRNA (0.1 nmol) in 60% fetal bovine serum in PBS was incubated at 370 for 0-48 h, then assessed on a 2% agarose gel in 1× TAE Buffer. Gels were stained with GelRed Nucleic Acid Stain (Biotium) according to the manufacturer's protocol.

Cryosectioning, Immunofluorescence, and Confocal Microscopy of Tumor Sections. Samples were snap frozen in OCT embedding medium and stored at −80° C. until processing for cryosectioning. Samples were serially sectioned until an adequate depth was reached for optimal visualization. Cryosections at various depths along the tumor were then sectioned at 6 μm thickness, captured, and placed on a slide. Slides were then processed immediately using ProLong Gold Antifade Mountant with DAPI with a cover slip or fixed in 4% paraformaldehyde for 10 minutes before immunofluorescence staining was done using a rabbit anti-firefly luciferase (anti-Fluc antibody, Abcam, ab185924; 1:500), a goat anti-rabbit Alexa Fluor® 488 (ab150077, Abcam; 1:500); Stained slides were counterstained with DAPI and imaged on a Nikon Eclipse Ti inverted confocal microscope. Imaging settings were kept constant across different treatment groups.

Biolayer Interferometry. Binding kinetics were measured by biolayer interferometry using an Octet RED 96 system (ForteBio). Duplexes synthesized with TEG-Biotin on the 5′ terminus of the antisense strand were diluted to 500 nM in Dulbecco's phosphate buffered saline containing calcium and magnesium (DPBS^{+/+}) and loaded on a Streptavidin Dip and Read Biosensor (ForteBio) for 600 sec. Baseline was then established over 120 sec in DPBS^{+/+} followed by association to either human or mouse serum albumin in DPBS^{+/+} over 300 sec. Subsequently, the biosensor was immersed in DPBS^{+/+} for 300 sec to measure dissociation. All steps were conducted at 30° C. and 1000 rpm. The binding values were measured using Octet Data Analysis HT Software. Reference biosensor values (biotinylated conjugate bound with no analyte) were subtracted to account for signal background. Y axes were aligned to the average of the baseline step. Interstep correction was performed by aligning to the dissociation step, and noise filtering was performed. Global analysis was performed to derive constants simultaneously from all tested analyte concentrations.

CriticalMicelle Concentration. A serial dilution of duplexes was prepared in a 96-well plate from 20 μM to 10 nM in 50 μL of DPBS (Ca²⁺/Mg²⁺ free). Nile Red (1 μL of a 0.5 mg/mL stock solution) was added to each well. Samples were then incubated in the dark with agitation at 37° C. for 2 h, and fluorescent intensity was measured on a plate reader (Tecan) at excitation 535±10 nm and emission 612±10 nm. The critical micelle concentration was defined as the intersection point on the plot of the two linear regions of the Nile red fluorescence versus the duplex concentration.

GelMigration Shift Experiments. Binding of siRNA conjugates (0.1 nmol) to human or mouse serum albumin (in 5× molar excess) incubated for 30 min at 37° C. was assessed by migration through 4%-20% polyacrylamide gels (Mini-Protean TGX). siRNA was visualized with GelRed Nucleic Acid Stain (Biotium) for ultraviolet imaging, and proteins were visualized with Coomassie blue and visible light imaging.

Conjugation efficacy of DBCO-modified siRNA duplex with azide-modified albumin was visualized using the Agilent Protein 230 Assay on the Agilent 2100 Bioanalyzer according to manufacturer instructions.

Long-Term Fluorescence ofBlood Samples. Longer term pharmacokinetic profiles of siRNA conjugates were established by measuring fluorescence of blood samples taken at various time points from 5 min to 24 h. Blood was sampled in the contralateral vein from injection (˜10 μL) using EDTA coated capillary tubes and stored at −80° C. Blood was then thawed and diluted 40× with phosphate buffered saline in a 96-well plate and fluorescent intensity was measured.

Intravital Microscopy and Biodistribution. Microscopy was performed using a Nikon Czsi+system. Isoflurane-anesthetized, 6-8 week old male CD-1 mice (Charles River) were immobilized on a heated confocal microscope stage for ear vein imaging. Mouse ears were depilated and then immobilized on a glass coverslip using microscope immersion fluid. Ear veins were detected using light microscopy, and images were focused to the plane of greatest vessel width, where flowing red blood cells were clearly visible. Once in focus, confocal laser microscopy was used to acquire one image per second, at which point Cy5-labeled siRNA (1 mg/kg) in 100 μL was delivered via tail vein. Fluorescent intensity within a circular region of interest (ROI), drawn in the focused vein, was used to measure fluorescence decay. Values are normalized to maximum initial fluorescence and fit to a one-compartment model in PK Solver to determine pharmacokinetic parameters.

Approximately 45 min after delivery of Cy5-labeled siRNA, blood was collected by cardiac puncture using EDTA-coated tubes and used for plasma isolation. Cy5 fluorescence was quantified in heart, lung, liver, kidney, and spleen using IVIS Lumina Imaging system (Xenogen Corporation) at excitation and emission wavelengths of 620 and 670 nm, respectively, using Living Image software version 4.4.

Size Exclusion Chromatography (SEC). Murine plasma was filtered (0.22 μm) then injected into an AKTA Pure Chromatography System (Cytiva) with three inline Superdex 200 Increase columns (10/300 GL) for fractionation at 0.3 mL/min using Tris running buffer (10 mM Tris-HCl, 0.15M NaCl, 0.2% NaN₃) into 1.5 mL fractions with a F9-C 96-well plate fraction collector (Cytiva). Cy5 fluorescence was measured in fractions (100 μL) in black, clear-bottom, 96-well plates (Greiner-Bio-one REF 675096) on a SynergyMx (Biotek) at a gain of 120, excitation 642/9.0, emission 675/9.0. Fraction albumin-bound conjugate was determined by taking the sum of fluorescence intensity for fractions associated with albumin elution divided by the sum of fluorescence intensity for all fractions collected. Albumin-associated fractions were determined by running known protein standards through the SEC system and examining A280 of eluent from each of the fractions.

Orthotopic Mammary Tumor Studies. Luciferase-expressing MDA-MB-231 cells (1×10⁶) in 100 μL of 50% Matrigel were injected into the inguinal mammary fat pads of 4-6 week old female athymic Balb/C (nu nu) mice (Envigo). Mice were randomized into treatment groups when tumors reached 50 mm³. Mice were treated once every 2 days with 2.5 mg/kg (based on parent siRNA molecular weight) luciferase-targeting siRNA until day 7, for a total of 3 treatments. Approximately 18 h before sacrifice, mice were treated with 1 mg/kg Cy5-labeled, nonactive siRNA conjugate. Mice were euthanized and organs collected at necropsy for measurements of biodistribution (as described above) and for molecular histological analysis. Tumors were minced and either flash frozen in liquid nitrogen for downstream assays, embedded in OCT and frozen for sectioning, or allocated for flow cytometry.

In Vivo Tumor Cell Uptake. Minced tumor was washed with HBSS containing Ca²⁺ and Mg²⁺ and then agitated for 1 h in an enzyme mix containing collagenase (0.5 mg/mL, Roche Life Sciences) and DNase (0.19 mg/mL, BioRAD) in DMEM. Tumors were centrifuged and resuspended in HBSS without Ca²⁺ and Mg²⁺ and then incubated with 5 mM EDTA for 20 min. Tumors were then centrifuged, and the pellets were resuspended in HBSS with Ca²⁺ and Mg²⁺ and filtered using a 70 m Nylon cell strainer. Filtrate was then washed once more with HBSS containing Ca²⁺ and Mg²⁺ and then incubated in ACK lysis buffer (Thermo Fisher Scientific) for 2 min before being diluted in 20 mL of PBS−/−. Cells were then pelleted and resuspended in 1-2 mL PBS−/− prior to running on a flow cytometer (Guava easyCyte). Uptake analysis was performed in FlowJo. Cell populations were isolated using forward and side scatter, then GFP positive tumor cells were gated, and Cy5 fluorescence intensity was measured for the GFP positive tumor cell population.

In Vivo Tumor Gene Silencing. Tumor fragments (200-300 mg) were lysed for 1 h on ice with agitation in 1× Reporter Cell Lysis Buffer (Promega) and then centrifuged at 14,000×g for 15 min at 4° C. Protein concentration was then quantitated using BCA Assay (Pierce). Lysates (20 mg per well) were assessed in 96-well plates using 90 μl reconstituted Luciferase Assay Substrate (Promega) according to the manufacturer's directions. Luminescence was measured using IVIS grid quantitation.

MCLI mRNA was measured in MDA-MB-231 tumors using QuantiGene SinglePlex assay (Thermo Fisher). Tumors were harvested and stored in RNAlater (Thermo Fisher) at 4° C. Tumors were dissociated in RNAlater in GentleMACS C-tubes (Miltenyi Biotec), washed twice with water, then digested for 6 h at 55° C. in Quantigene Diluted Lysis Mixture (DLM) supplemented with proteinase K (0.25 mg/ml) using 2 mls DLM per gram of tumor. Tissue lysates were diluted 1:2 for Quantigene assessment with manufacturer-designed probe sets directed against human MCLI and human PPIB. Luminescence generated from each specific probe set was measured and quantified on a plate reader (Tecan). Each sample was assessed in 5 technical replicates. Values shown are the ratio of MCLI (corrected for the loading control, PPIB). All values shown are relative to the average MCLI level observed in tumors from saline-treated mice.

StatisticalAnalyses. Data were analyzed using GraphPad Prism 7 software (Graphpad Software, Inc.) Statistical tests used for each data are provided in the corresponding figure captions. For all figures, *p≤0.05**p≤0.01***p≤0.001****p≤0.0001. All plots show mean standard deviation.

### Example 2

Divalent Lipid Modifier Improves Bioavailability of Chemically Stabilized siRNAs

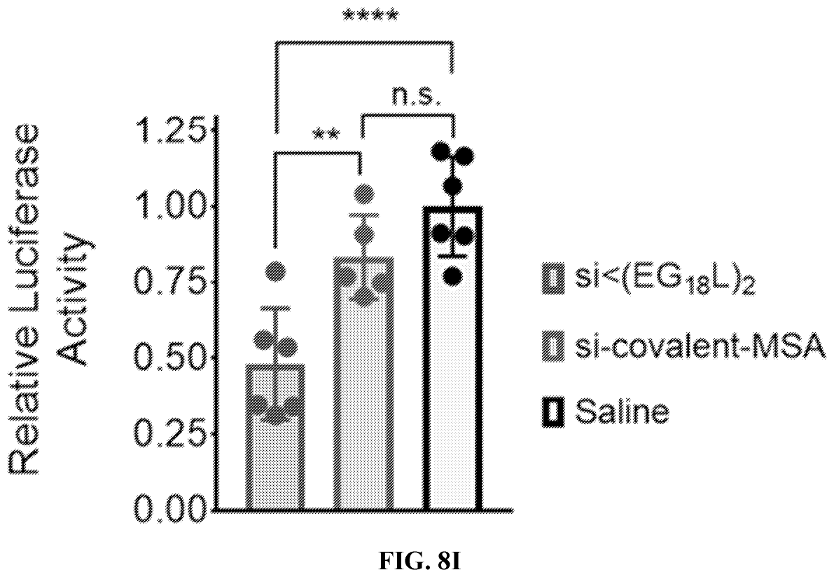

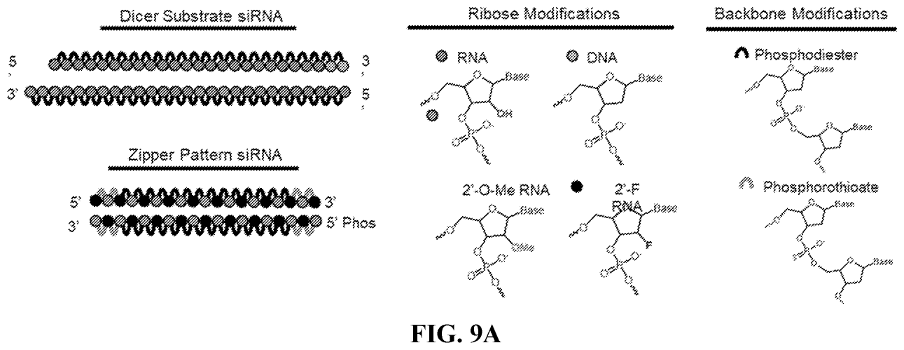

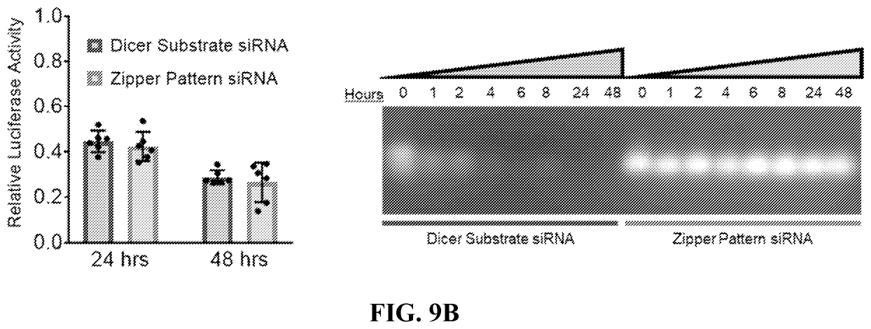

To finely tune and examine the structure-function relationship of siRNA variants, a library of siRNA-lipid conjugates was generated using solid phase synthesis, which maximizes product yield, purity, and reproducibility compared to previously reported two-step solution phase conjugation as described in Sarett, S. M. et al. Lipophilic siRNA targets albumin in situ and promotes bioavailability, tumor penetration, and carrier-free gene silencing. Proceedings of the National Academy of Sciences of the United States of America 114, E6490-E6497 (2017), which is incorporated herein by reference in its entirety. The synthesized siRNAs were designed to be fully stabilized with alternating 2′F and 2′OMe modifications in a “zipper” pattern and terminal phosphorothioate linkages (FIG. 9A). These modifications can confer endonuclease and exonuclease resistance. It was demonstrated that these stabilizing siRNA modifications maintain gene silencing potency and provide serum stability, while traditional Dicer substrate siRNAs are similarly potent but degrade within 4 h of serum challenge (FIG. 9B).

Valency may affect bioavailability and pharmacodynamics of lipid end-modified siRNA conjugates in vivo. Conjugation to one or two 18-carbon stearyls was focused on, an albumin-binding lipid with higher albumin affinity than those with shorter lipid chain lengths, for initial assessment of modifier valency on siRNA pharmacokinetics. Absolute circulation half-life (t_{1/2}) was measured using real-time fluorescence imaging of Cy5-labeled siRNA conjugates within mouse vasculature, revealing increased t_{1/2} (46±5.9 min) of siRNA conjugated to divalent (L₂) over monovalent (L₁) stearyl (28±4.2 min). Importantly, L₂-conjugated siRNA (siRNA-L₂) showed diminished kidney accumulation compared to siRNA-L₁, suggesting that renal clearance, the primary elimination mechanism of circulating siRNAs, is reduced with siRNA-L₂. The remainder of the studies, therefore, focused on a divalent lipid design.

### Example 3

### Hydrophilic Linker Length Increases In Vitro Albumin Affinity and Reduces Self-Assembly of Lipid-siRNA Conjugates

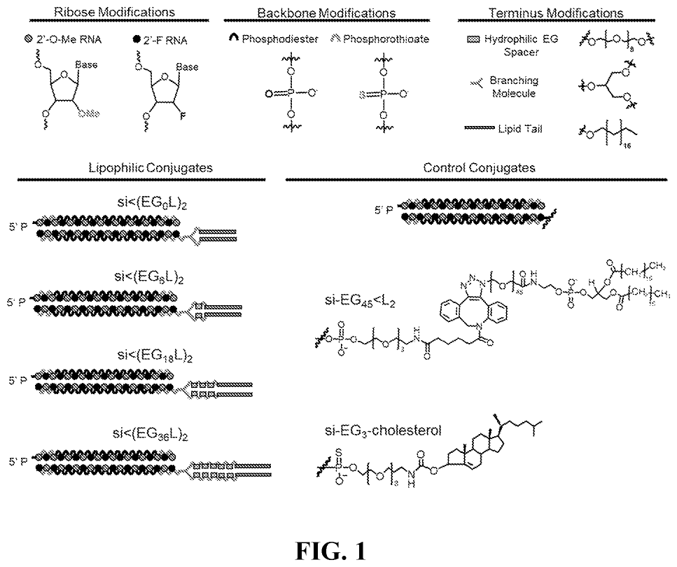

Based on the improved performance of siRNA-L₂ over siRNA-L₁ in vivo, siRNA-L₂ was modified to assess the functional effects of structural modification of the hydrophilic linker between the lipid and siRNAs. Specifically, the number of ethylene glycol (EG) repeats were progressively increased from no EG repeats [si<(EG₀L)₂] to 30 EG repeats [si<(EG₃₀L)₂]; the EG repeats were added in increments of 6, using a hexaethylene phosphoramidite (FIG. 1). Two previously described serum protein-binding siRNA conjugates, cholesterol-TEG-siRNA (si-chol) and si-EG₄₅<L₂, were synthesized as comparative references.

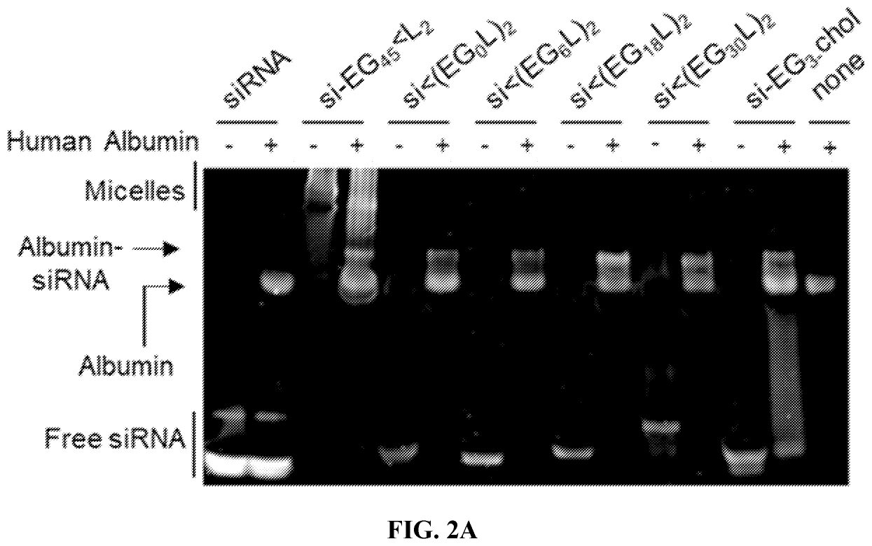

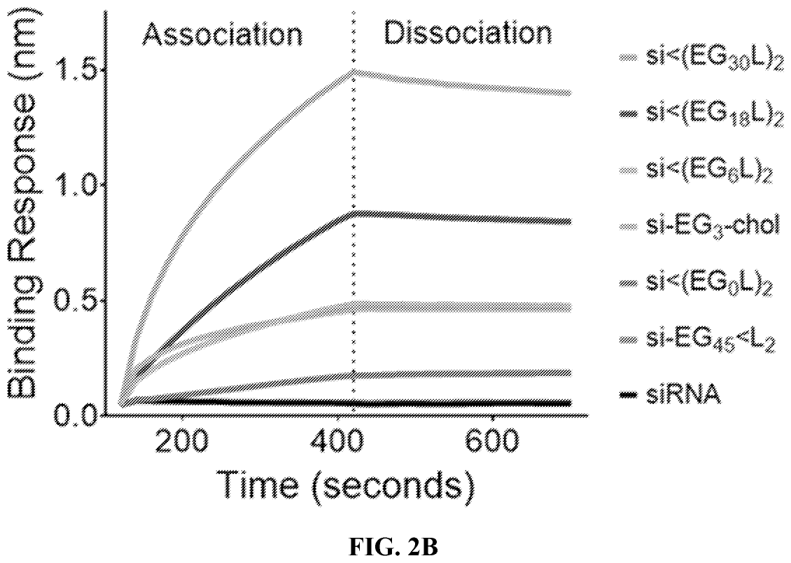

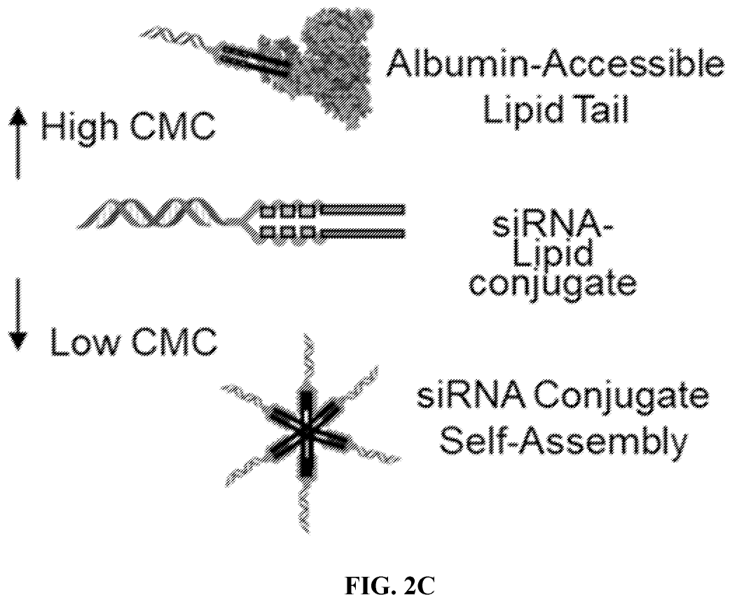

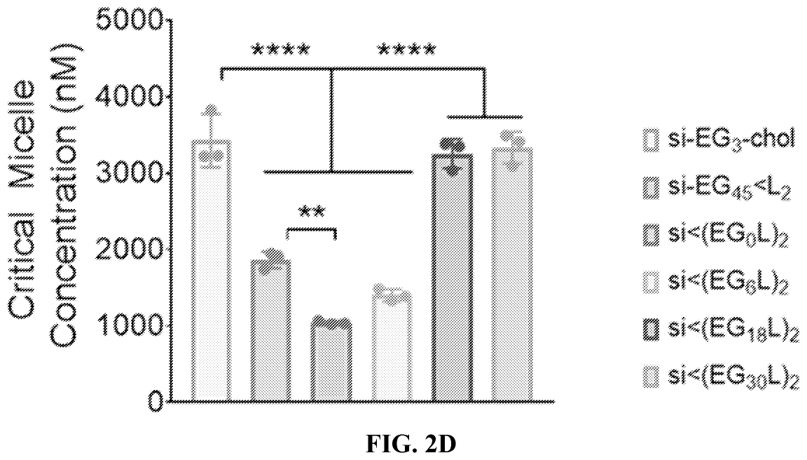

Each siRNA<(EGxL)₂ was incubated with human serum albumin to assess albumin-siRNA complex formation by electrophoretic mobility shift assay. These studies revealed that, while electrophoretic mobility of free siRNA was unaffected by albumin, the mobility si<(EG₀L)₂, si<(EG₆L)₂, si<(EG₁₈L)₂, and si<(EG₃₀L)₂ was lowered upon exposure to albumin (FIG. 2A), consistent with the high molecular weight of the complex formed by albumin and siRNA conjugates. Similarly, si-cholesterol, and si-EG₄₅<L₂ also displayed albumin-dependent mobility shifts in this assay. However, super-shifting of si-EG₄₅<L₂ was seen in both the presence and absence of albumin, suggesting that si-EG₄₅<L₂ may harbor some self-association properties which were not seen in si<(EGxL)₂ conjugates. Similar results were observed using mouse serum albumin.

Albumin association and dissociation kinetics of the si<(EGxL)₂ variants were studied further using biolayer interferometry (FIG. 2C). Free siRNA did not exhibit binding with albumin, while si-cholesterol exhibited moderate albumin binding. Although si<(EG₀L)₂ displayed decreased HSA binding response compared to si-cholesterol, siRNA conjugates harboring a greater number of EG spacers within the linker element had progressively higher affinity for HSA, with both si<(EG₁₈L)₂ (K_{D}=30±0.3 nM) and si<(EG₃₀L)₂ (K_{D}=9.49±0.1 nM) exhibiting higher affinity albumin binding than si-cholesterol and si-EG₄₅<L₂. The substantial difference in albumin binding response between variants underscores the role of the EG repeats within the linker region.

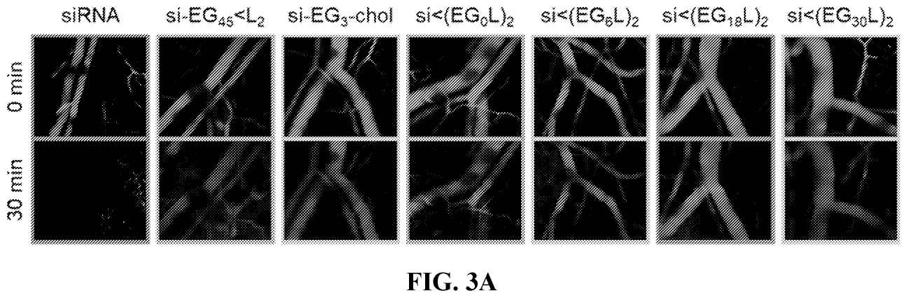

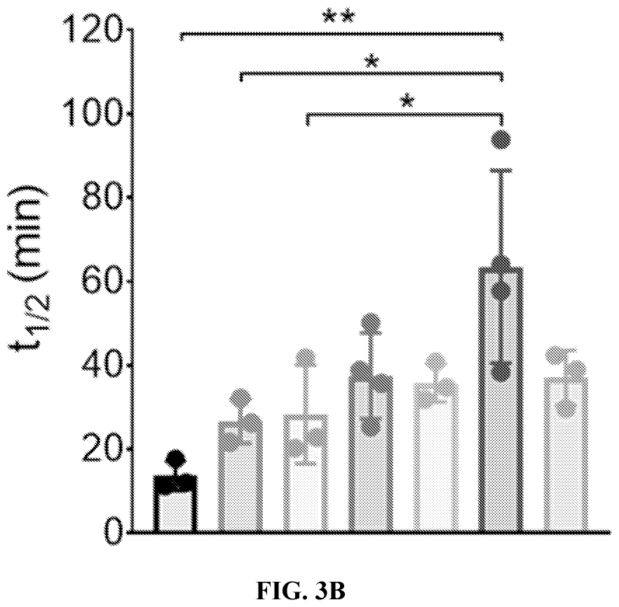

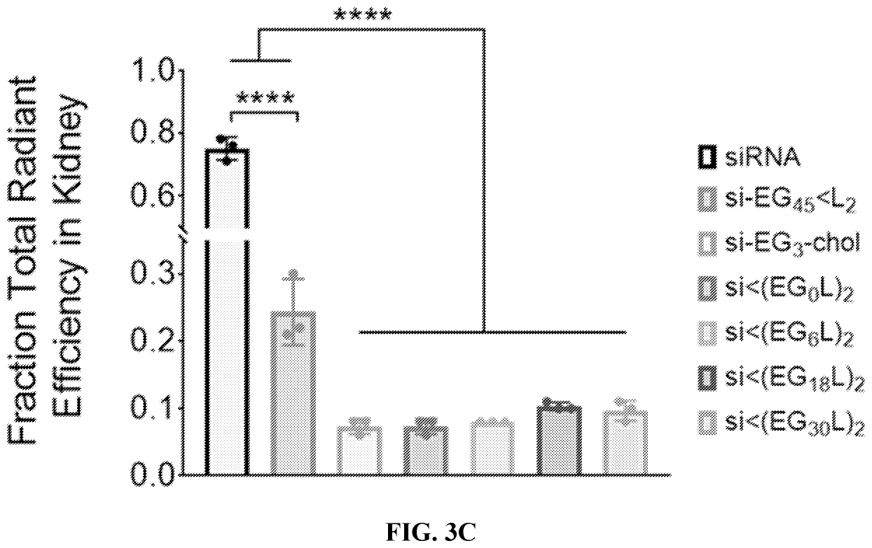

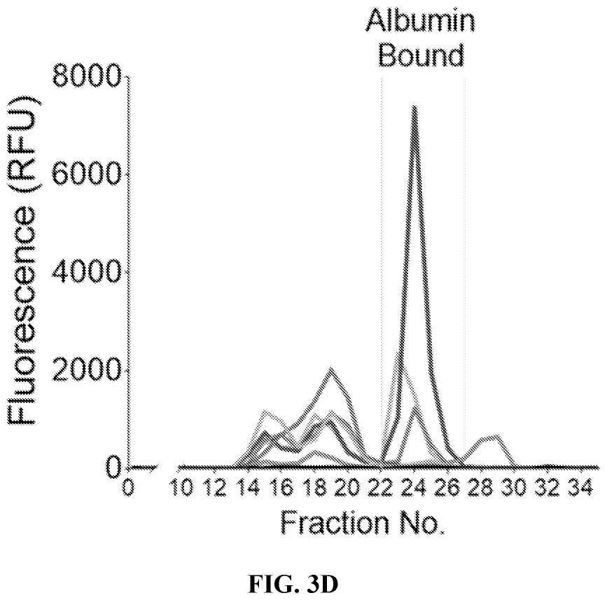

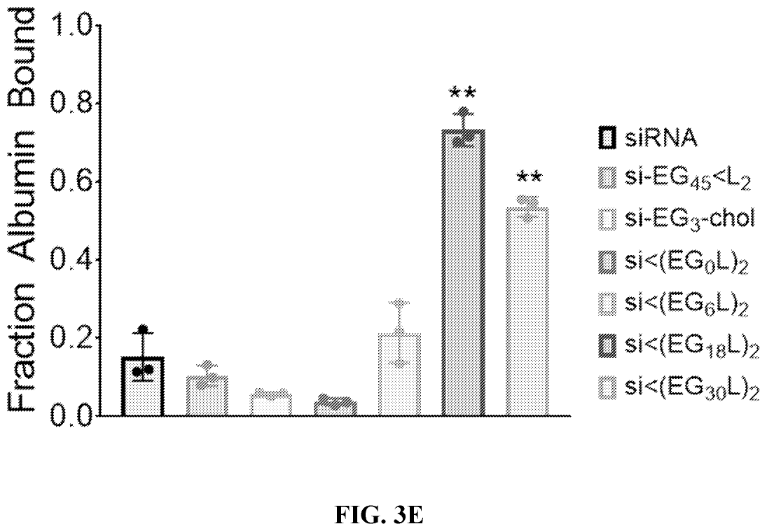

Amphiphilic lipid-modified nucleic acids have a tendency to self-assemble into micellar structures, particularly when using long lipid chains. It is possible that self-aggregation of amphiphilic siRNA-lipid conjugates is a competing interaction that might interfere with albumin association, particularly if lipid tails can become sequestered in the core of a self-assembled structure, where they would be rendered unavailable for interaction with the fatty acid binding pockets of albumin (FIG. 3C). Thus, the critical micelle concentration (CMC) was determined for each si<(EGxL)₂ to establish the impact of linker length on siRNA conjugate self-assembly (FIG. 2D). The si-cholesterol exhibited a relatively high CMC (3430±350 nM), suggesting a low tendency for si-cholesterol to self-associate. This is consistent with the bulky structure of cholesterol, which is not amenable to close packing like lamellar long-chain lipids. In contrast, si-EG₄₅<L₂ exhibited a lower CMC (1860±60 nM), indicating a higher tendency towards self-association. Interestingly, si<(EG₀L)₂, which lacks any EG spacer in the linker element, exhibited the lowest CMC (1040±23 nM) and thus the highest propensity for self-association, while the increased number of EG repeats in si<(EG₁₈L)₂ and si<(EG₃₀L)₂ correlated with the highest CMCs (3260±190 nM and 3330±210 nM), and thus the lowest tendency towards self-association.

### Example 4

### Hydrophilic Linker Length Influences Pharmacokinetics and In Vivo Plasma Disposition of Lipid-siRNA Conjugates

The structure of hydrophobic modifications on siRNA can be tuned to direct siRNA binding to different serum components, such as lipoproteins and albumin, after intravenous administration, which can consequently modify pharmacokinetics and biodistribution. Although there is evidence for active uptake of both albumin and lipoproteins by tumor cells, it was sought to preferentially bind to albumin because of its smaller size and lower relative liver tropism. The in vivo half-life of human albumin is approximately 19 days, making it a good candidate for improving the pharmacokinetics of candidate therapeutics.

Each of the siRNA conjugates was intravenously administered to mice to understand how EG repeats within the linker affect conjugate pharmacokinetics. Intravital fluorescence microscopy of Cy5-labeled siRNA conjugates flowing through vessels of the mouse ear demonstrated the rapid and complete diminution of circulating free siRNA within the first 30 min post-treatment, while serum component-binding si-cholesterol and si-EG₄₅<L₂ retained some observable circulating siRNA (FIG. 3A). Interestingly and unexpectedly, increased EG repeats correlated with increased retention of circulating siRNA in si<(EGxL)₂ conjugates to a point, but once the linker became too long (e.g., si<(EG₃₀L)₂), retention was reduced, with si<(EG₁₈L)₂ showing maximal circulation retention of the variants tested. Real-time collection of vascular fluorescence imaging data throughout the first hour post-treatment enabled calculation of absolute half-life (t_{1/2} abs), demonstrating the substantially prolonged t_{1/2} abs of si<(EG₁₈L)₂, which was greater than what was observed for si<(EG₃₀L)₂ (FIG. 3B), and nearly 5 times that of si<(EG₀L)₂ (Table 1). Approximately 45 min after injection, there was significantly more renal accumulation, the primary clearance path for oligonucleotide-based therapeutics, of the parent siRNA and control conjugate, si-EG₄₅<L₂ (FIG. 3C).

TABLE 1
Pharmacokinetic parameters for siRNA conjugate
library determined from intravital microscopy.

| | | t_{1/2} | AUC_{0-35 m} | CL |
|---|---|---|---|---|
| | | (min) | (μ · min)/(mL) | (mL)/(min) |
| | siRNA | 14 ± | 210 ± | 0.069 ± |
| | | 3.5 | 25 | 0.012 |
| EG | si-EG₃-cholesterol | 28 ± | 410 ± | 0.025 ± |
| Variants | | 12 | 42 | 0.0079 |
| | si-EG₄₅ < L₂ | 27 ± | 390 ± | 0.026 ± |
| | | 5.3 | 25 | 0.0046 |
| | si < (EG₀L)₂ | 33 ± | 416 ± | 0.028 ± |
| | | 6.6 | 55 | 0.0059 |
| | si < (EG₆L)₂ | 36 ± | 450 ± | 0.019 ± |
| | | 4.6 | 15 | 0.0022 |
| | si < (EG₁₈L)₂ | 64 ± | 470 ± | 0.012 ± |
| | | 23 | 52 | 0.0032 |
| | si < (EG₃₀L)₂ | 37 ± | 440 ± | 0.019 ± |
| | | 6.6 | 36 | 0.0043 |
| PS | si < (EG₁₈L)₂No 5′Se | 37 ± | 430 ± | 0.020 ± |
| Variants | PS | 12 | 50 | 0.0060 |
| | si < (EG₁₈L)₂No 5′Se | 15 ± | 300 ± | 0.047 ± |
| | | 1.5 | 36 | 0.0070 |
| Branching | si-(EG₁₈) < L₂ | 67 ± | 490 ± | 0.012 ± |
| Variants | | 32 | 35 | 0.0070 |
| | si-(EG₃₆) < L₂ | 41 ± | 450 ± | 0.018 ± |
| | | 13 | 27 | 0.0050 |
| Lipid | si < (EG₁₈L_{diacid})₂ | 47 ± | 469 ± | 0.017 ± |
| Variants | | 21 | 64 | 0.0073 |
| | si < (EG₁₈L_{unsaturated})₂ | 34 ± | 391 ± | 0.029 ± |
| | | 26 | 121 | 0.016 |

It is hypothesized, without being bound by a particular theory, that the diminished renal clearance of the si<(EGxL)₂ variants over si-EG₄₅<L₂ is due to the presence of hydrolytically degradable ester bonds located in the structure of DSPE-PEG₂₀₀₀ used to make si-EG₄₅<L₂. Albumin itself is known to possess intrinsic esterase activity, making the hydrolytic stability of drugs that interact with it particularly important. Hydrolysis of this ester would be expected to release the siRNA from albumin, possibly prematurely in the circulation, resulting in renal clearance and shorter circulation time that is more analogous to the non-modified parent siRNA structure. Conjugates that remain albumin-bound, by comparison, can evade renal clearance through albumin's natural reabsorption in the renal proximal tubule where, after endocytosis by the megalin-cubilin complex, the neonatal Fc receptor redirects albumin and its associated cargo back to the interstitial space, facilitating its return to the circulation via the lymphatics.

Plasma was collected from mice treated intravenously with Cy5-labeled siRNA conjugates and analyzed by size exclusion chromatography to measure the level of each candidate's association with albumin versus other plasma fractions (e.g., lipoproteins) (FIG. 3D). Cy5 fluorescence was detected in albumin-containing plasma fractions, as well as in fractions not representative of albumin elution. Notably, plasma isolated from mice treated with si<(EG₁₈L)₂, the most long-circulating on the investigated conjugates, exhibited the most robust peak within the albumin-containing fraction at approximately 75% bound (FIG. 3E). This observation is consistent with the BLI data showing the high affinity of si<(EG₁₈L)₂ with albumin and also suggests a positive correlation between the percent of conjugate that is albumin-bound in vivo and the circulation half-life.

It is interesting and unexpected that in vitro albumin binding affinity analyses indicate that si<(EG₃₀L)₂ binds albumin more favorably than si<(EG₁₈L)₂ (FIG. 2B), while albumin association in vivo is greater with si<(EG₁₈L)₂. This may be attributable to the larger entropic penalty of binding incurred for this larger and more flexible molecule that is mitigated by the ideal conditions of in vitro testing but becomes more apparent in vivo.

### Example 5

Albumin-Binding Increases Tumor Accumulation and Carrier-Free Silencing of siRNA-Lipid Conjugates

The observed positive correlation between hydrophilic linker length and albumin-bound siRNA in vivo enabled interrogation of potential correlations between conjugate albumin association and tumor accumulation. Luciferase (Luc)-expressing MDA-MB-231 triple negative breast cancer cells were injected into the inguinal mammary fat pad to generate orthotopic tumors in female athymic mice. When tumors exceeded 50 mm³, candidate siRNAs and controls were delivered i.v. at 2.5 mg/kg on days 1, 3, and 5. On day 6, Cy5-labeled si<(EGxL)₂ was delivered at 1 mg/kg, and Cy5 distribution to tumors and organs (kidney, liver, spleen, heart, lungs) were assessed on day 7, approximately 18 h after final treatment.

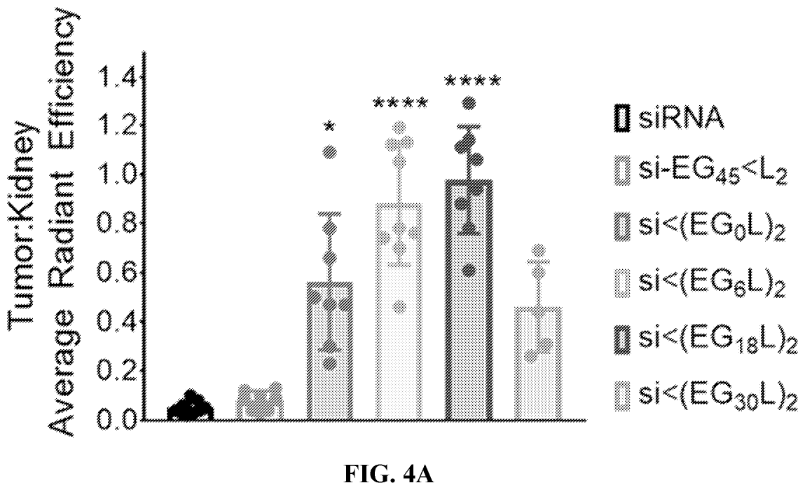

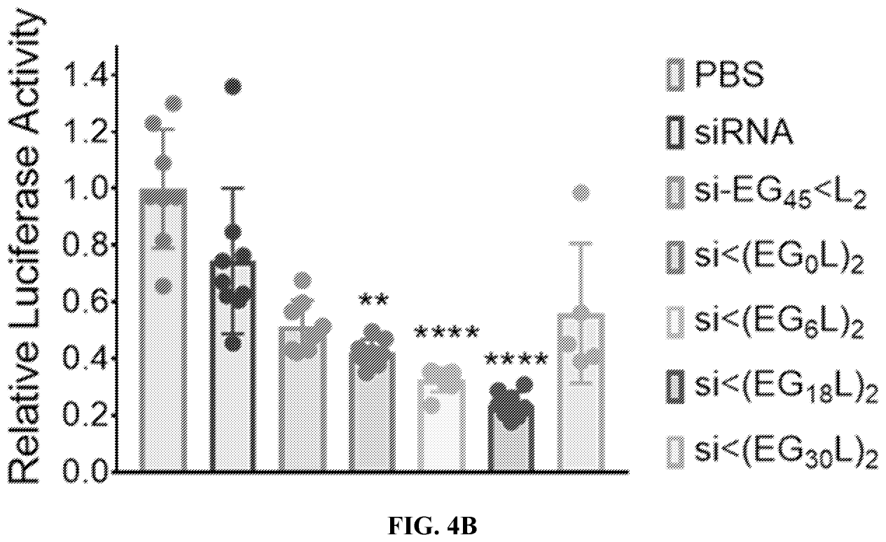

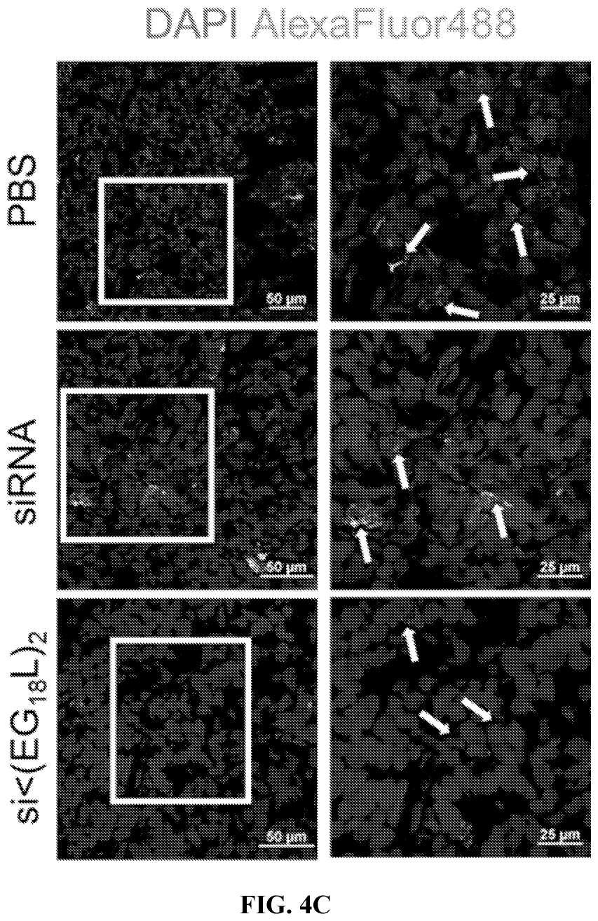

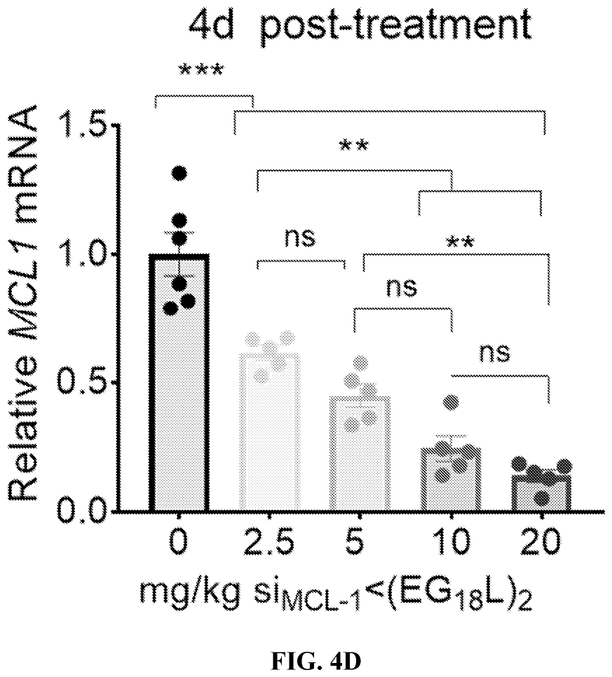

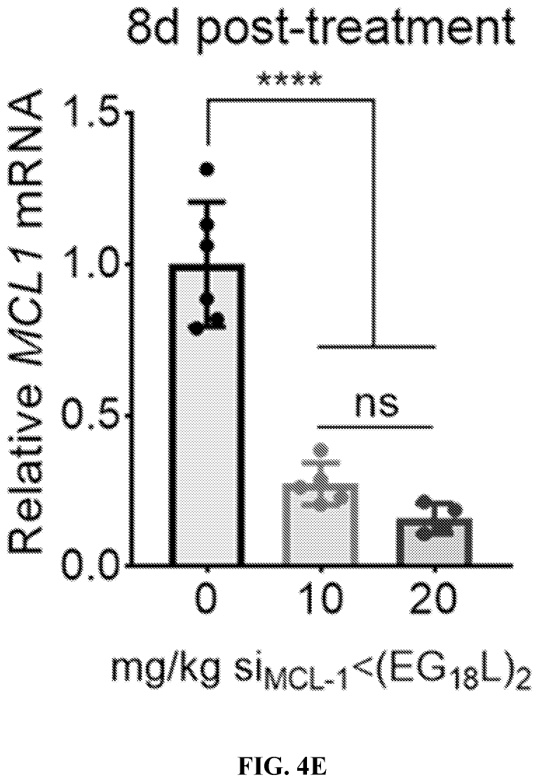

Free Cy5-siRNA largely accumulated in the kidney, but not the tumor, resulting in a tumor to kidney fluorescence ratio of less than 0.05 (FIG. 4A), which was modestly increased in si-EG₄₅<L₂ treated mice. However, progressively increased tumor fluorescence along with progressively decreased kidney fluorescence was observed in mice treated with si<(EG₀L)₂, si<(EG₆L)₂, and si<(EG₁₈L)₂, resulting in a tumor to kidney fluorescence ratio of 0.5, 0.9, and 1.0, respectively, suggesting that increased linker length affected siRNA tumor accumulation. These results are consistent with the trends for albumin binding and circulation time seen across the library of si<(EGxL)₂ constructs, since longer circulating siRNA conjugates can enable higher tumor exposure and interaction with target cells. Interestingly, tumor to kidney fluorescence was lower in mice treated with si<(EG₃₀L)₂ compared to other si<(EGxL)₂ candidates. This result reinforces the indication that, despite high affinity binding to purified albumin, si<(EG₃₀L)₂ does not perform as well in the more complex in vivo environment compared to candidates with a shorter linker. These results also support the broader conclusion that diminished albumin association in plasma following intravenous delivery leads to lower tumor accumulation.

Strikingly, at the end of the treatment regime, mice treated with si<(EG₁₈L)₂ targeting luciferase transgene showed nearly 80% reduction in tumor cell luciferase activity compared to mice treated with a sham control (FIG. 4B). Indeed, si<(EG₁₈L)₂ also shows more than twice the silencing potency si-EG₄₅<L₂. Immunohistochemistry of tumors stained for luciferase protein further demonstrated robust silencing by si<(EGi₈L)₂ throughout sections compared to saline and unmodified siRNA controls (FIG. 4C). Prior reports of carrier-free siRNA conjugates for tumor delivery are believed not to have achieved this level of silencing, even at higher doses. For instance, cholesterol-siRNA administered at 10 mg/kg i.v. was reported to achieve approximately 50% silencing (Chernikov, I. V. et al. Cholesterol-Containing Nuclease-Resistant siRNA Accumulates in Tumors in a Carrier-free Mode and Silences MDR1 Gene. Molecular Therapy—Nucleic Acids 6, 209-220 (2017), which is incorporated by reference herein in its entirety) in xenograft tumors, and a receptor-targeted siRNA fused with a Centyrin ligand that was administered 3×10 mg/kg reduced target mRNA expression by approximately 60% (Klein, D. et al. Centyrin ligands for extrahepatic delivery of siRNA. Mol Ther 29, 2053-2066 (2021), which is incorporated herein by reference in its entirety). Thus, the unprecedented silencing potency achieved with a modest dose of si<(EG₁₈L)₂ motivated further interrogation of the structure-function of this candidate.

Conjugate si<(EG₁₈L)₂ was then synthesized to target endogenous oncogene MCL-1. For added rigor, a single bolus was administered intravenously to mice bearing MDA-MB-231 tumors. Four days after treatment, relative MCL-1 mRNA levels showed dose-dependent silencing, with 10 and 20 mg/kg injections achieving approximately 75% and 85% knockdown, respectively (FIG. 4D). This prompted the examination of whether these higher doses could achieve sustained knockdown at a later time point as the tumor cells continued to proliferate. These two higher doses were administered and assessed relative MCL-1 knockdown levels at 8 d post-treatment, which, strikingly, showed that the knockdown was not significantly diminished. These results suggest that the candidate holds promise as a therapeutic candidate that does not need to be administered more than once per week, as many current chemotherapies do.

### Example 6

### Phosphorothioate Linkages of Lipid-Modified Terminus Improves Conjugate Performance In Vitro and In Vivo

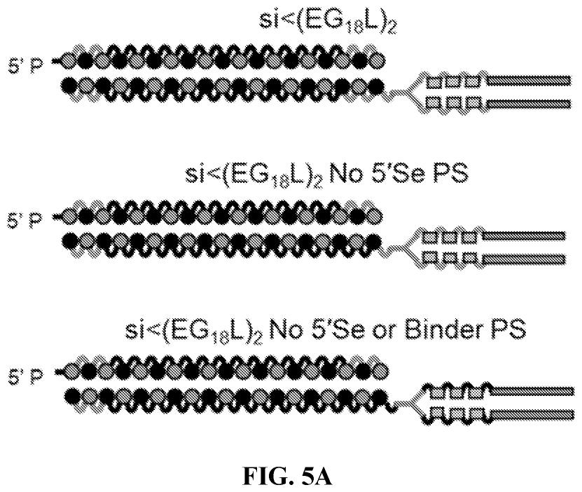

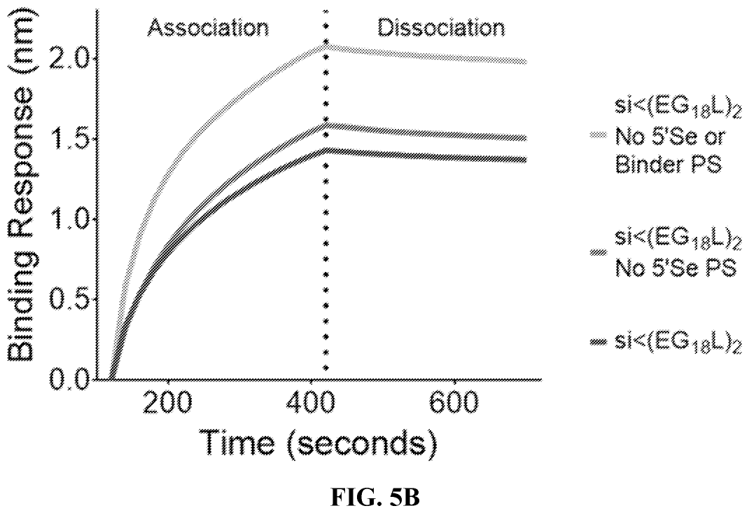

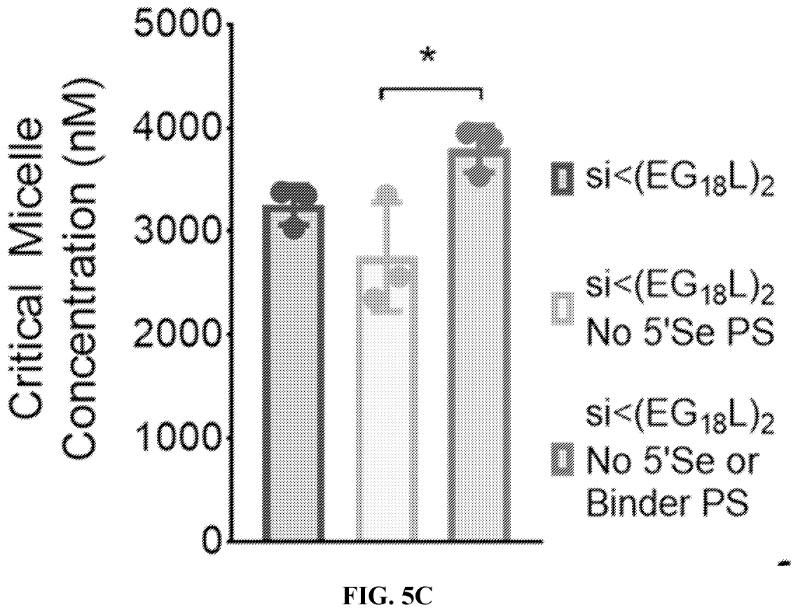

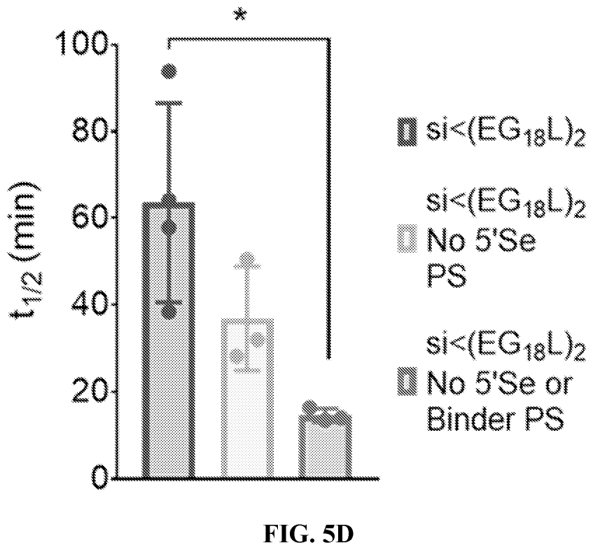

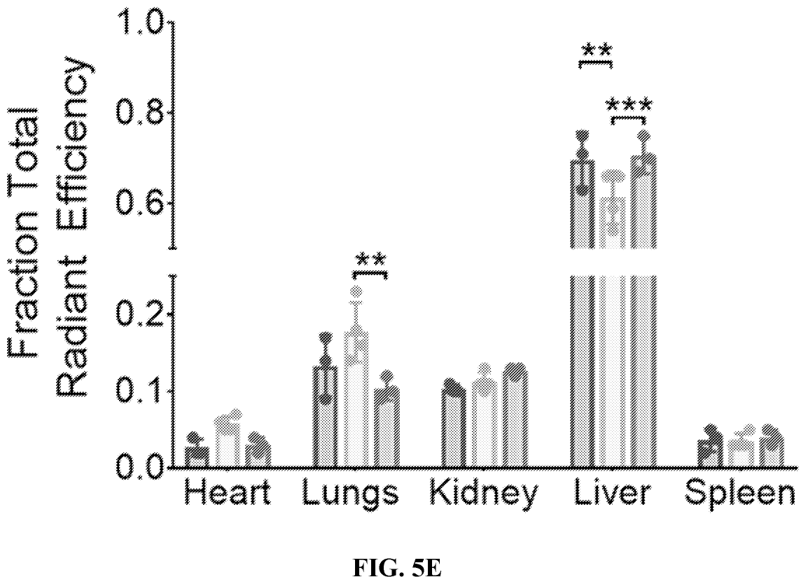

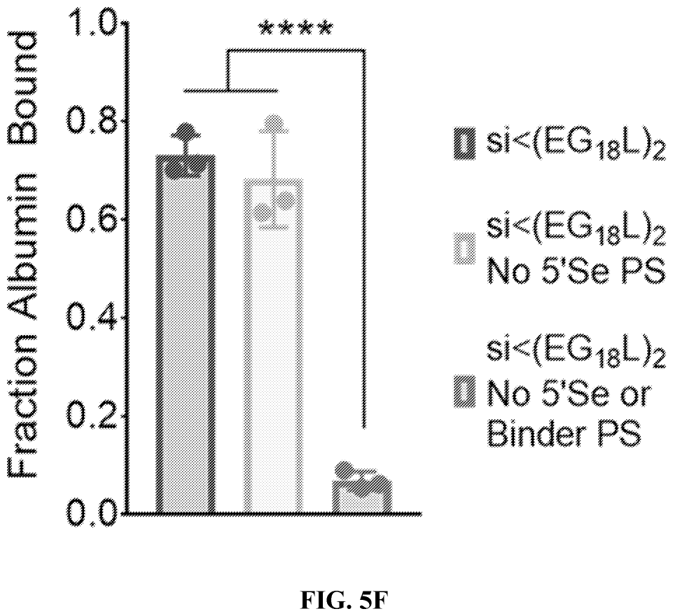

Deeper structural interrogation of si<(EG₁₈L)₂ was next focused on by assessing the impact of the phosphorothioate (PS) bonds at the 5′ sense terminus and between the EG₆ repeating units of the linker. Terminus stabilization with PS linkages in lieu of phosphodiester (PO) linkages has significant effects on performance of siRNA-based therapeutics by conferring exonuclease resistance and can help enable extrahepatic, carrier-free gene silencing applications. Variants of si<(EG₁₈L)₂ with PS bonds removed from the 5′ sense (Se) terminus (si<(EG₁₈L)₂ No 5′Se PS) or removed from both the 5′ sense terminus and each of the bonds in the linker to the stearyl groups (si<(EG₁₈L)₂ No 5′Se or Binder PS) (FIG. 5A) were synthesized and studied using biolayer interferometry to determine albumin binding kinetics (FIG. 5B). Both variants exhibited comparable albumin affinity as the parent construct with K_{D} values only varying ±2 nM. However, removing the PS bonds from the linker significantly increased the critical micelle concentration compared to just removing it from the 5′ sense terminus (2755±526 to 3798±225 nM), suggesting a lower tendency to self-assemble without the more hydrophobic PS bonds located on the linker (FIG. 5C).

Real-time, intravital microscopy of fluorescently labeled conjugates in circulation was performed to determine the effect of PS content on circulation time. Removal of PS bonds from both the 5′ sense terminus and the 5′ sense terminus/binder resulted in significantly diminished pharmacokinetic profiles compared to parent construct si<(EG₁₈L)₂ (FIG. 5D) with circulation half-lives reduced to 37±12 min and 15±1.5 min respectively from 64±23 min. Biodistribution of conjugates approximately 45 min after treatment demonstrated significant differences in PS-dependent accumulation in both the lungs and the liver (FIG. 5E). Plasma collected from mice treated intravenously with each siRNA conjugate to examine proteins bound in vivo demonstrated 10-fold lower relative albumin binding by si<(EG₁₈L)₂ siRNA conjugate when the PS bonds were not used at the 5′ sense terminus (FIG. 5F). These combined data suggest that the PS bonds within the linker facilitate albumin binding. The diminished pharmacokinetics observed with the removal of phosphorothioate bonds is most likely due to reduced albumin association and not due to degradation. This conclusion is supported by the observation that, unlike ester-containing si-EG₄₅<L₂, the kidney accumulation of the conjugate without PS bonds was similar to the fully modified parent construct.

### Example 7

Position of Branching Point in Divalent Lipid Conjugate affects Conjugate Performance In Vitro and In Vivo

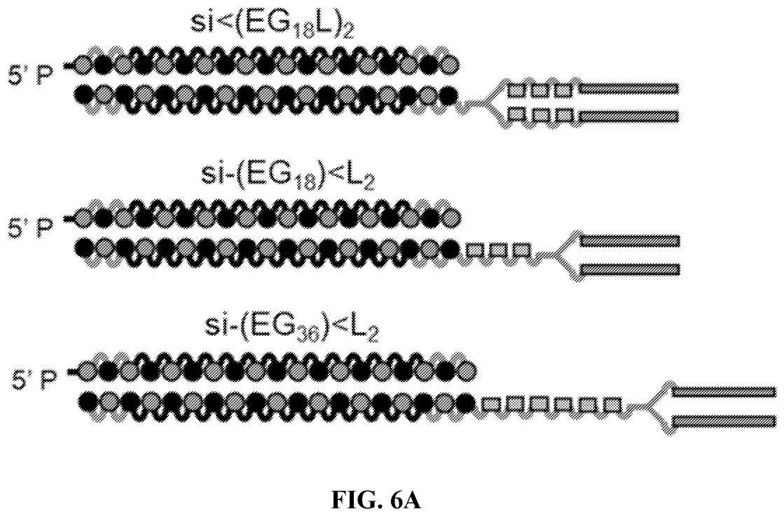

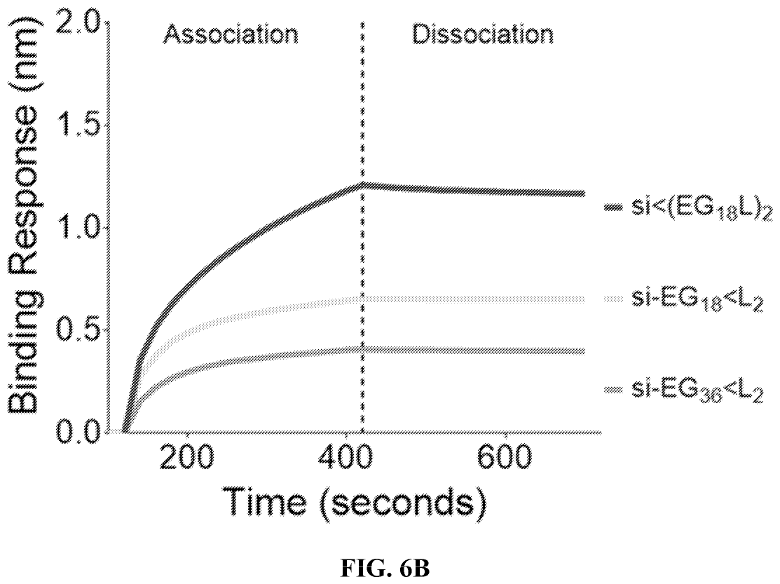

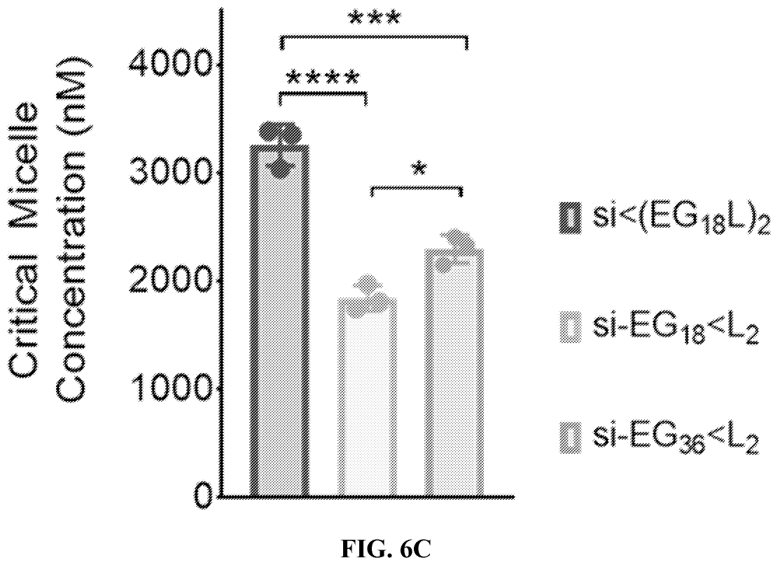

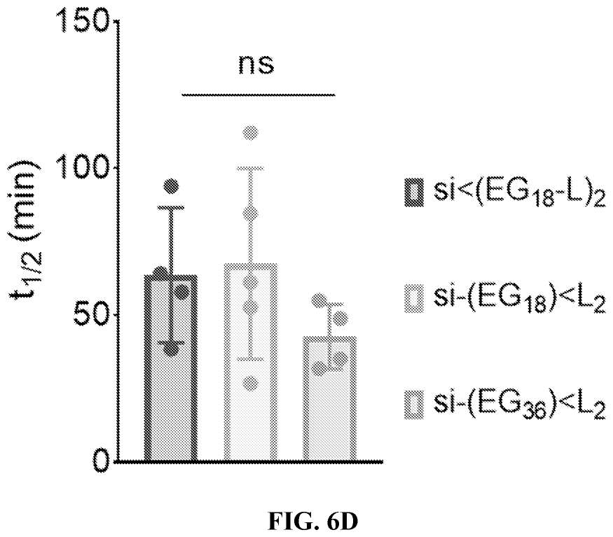

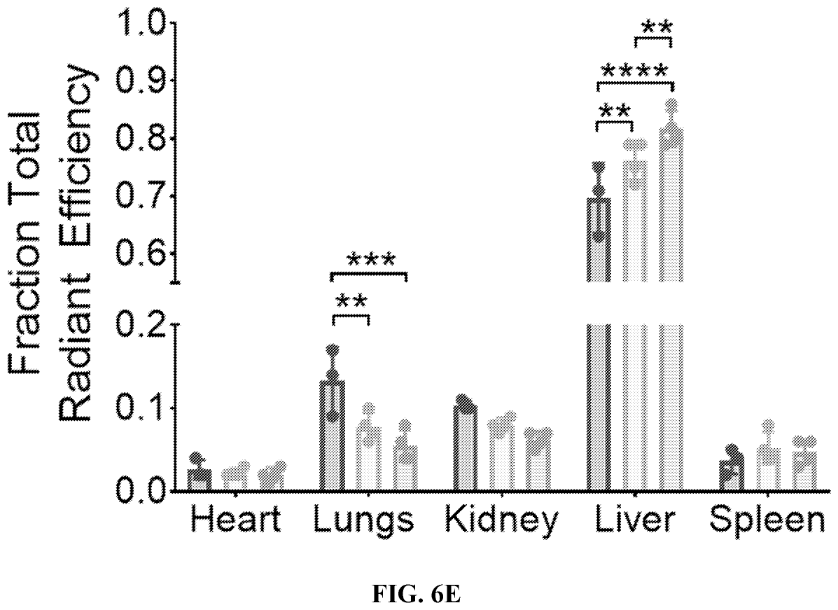

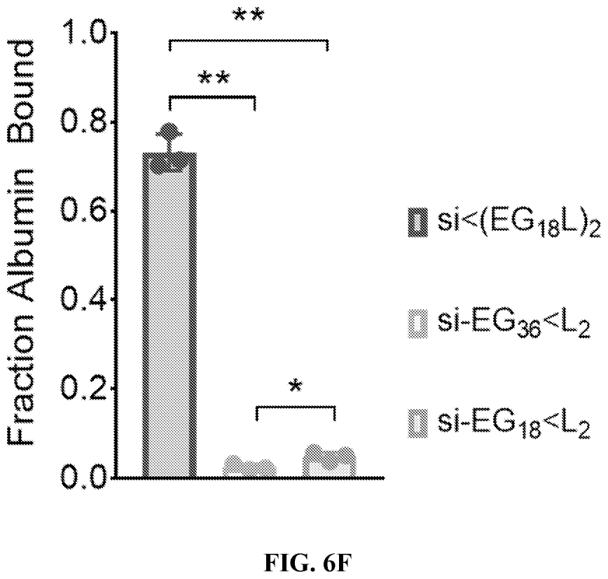

It was sought to determine whether the position of the branching point in the divalent si<(EG₁₈L)₂ conjugate influences candidate function. This study was motivated by the desire to further confirm the observation that albumin association is driven by reduced tendency to self-assemble rather than simply the relative level of conjugate hydrophilic linker content or linker length. It is hypothesized, without being bound by a particular theory, that placement of the branching point distal to the repeating EG linker and immediately proximal to the stearyl groups increases apparent hydrophobicity and consequent self-assembly by constraining the stearyl groups to remain tightly packed together. Since the greatest siRNA circulation time and tumor accumulation was seen with the si<(EG₁₈L)₂ conjugate, which correlated with increased albumin binding in vivo, two additional iterations of si<(EG₁₈L)₂ were generated with a distal branching location, one matched for overall ethylene glycol content (si-EG₃₆<L₂) and one matched for the distance between the siRNA and its lipid tail (si-EGis<L₂) (FIG. 6A). Biolayer interferometry measurement of albumin binding in vitro interestingly showed that both new branching point variants had lower albumin binding affinity relative to the parent construct si<(EG₁₈L)₂ (FIG. 6B). The CMC was measured by Nile Red encapsulation and both si-EG₁₈<L₂ and si-EG₃₆<L₂, similarly to control construct si-EG₄₅<L₂, exhibited significantly lower values (1838±117 and 2293±132 nM) compared to si<(EG₁₈L)₂ (3255±192 nM) (FIG. 6C). These data suggest that the more proximal branch site increases albumin association and consequent siRNA tumor activity at least partially due to decreased tendency of amphiphilic siRNA conjugates with this feature to self-assemble.

Intravital microscopy of circulating fluorescent conjugates showed that there was not a significant difference in absolute circulation half-life among the branching architecture variants (FIG. 6D). However, biodistribution of the fluorescently labeled conjugates approximately 45 min after administration revealed significantly higher liver accumulation of si-EGis<L₂ and si-EG₃₆<L₂ compared to si<(EG₁₈L)₂ (FIG. 6E). This observation may be explained by a tendency to bind a greater fraction of lipoproteins over albumin in vivo which can preferentially traffic the conjugates to the liver. Indeed, plasma isolated from mice treated with the conjugates and analyzed by SEC for associated proteins demonstrated significantly decreased albumin binding among branching architecture variants si-EGis<L₂ and si-EG₃₆<L₂ (˜5%) compared to si<(EG₁₈L)₂ (˜75%) (FIG. 6F). In sum, these data show that the distal placement of the branching point increases self-assembly and association with lipoproteins at the cost of diminished association with and piggybacking upon serum albumin.

### Example 8

### Hydrophobicity of Lipid is More Important Than Binding Affinity for Conjugate Performance

It was next sought to investigate whether the nature of the C₁₈ lipid itself has important implications in conjugate performance. To this end, two variants were synthesized—one with the carboxyl terminal still intact (si<(EG₁₈L_{diacid})₂) and one with a double bond (si<(EG₁₈L_{unsaturated})₂). The carboxyl handle of fatty acids is usually consumed in conjugation reactions. However, the development of GLP-1 agonist drug Semaglutide demonstrated the importance of this moiety for albumin-binding drugs. Indeed, by restoring the carboxyl on their lipid-peptide, they found significant increases in albumin affinity and circulation half-life. Thus, it was sought to explore whether having this group in this system would improve performance.

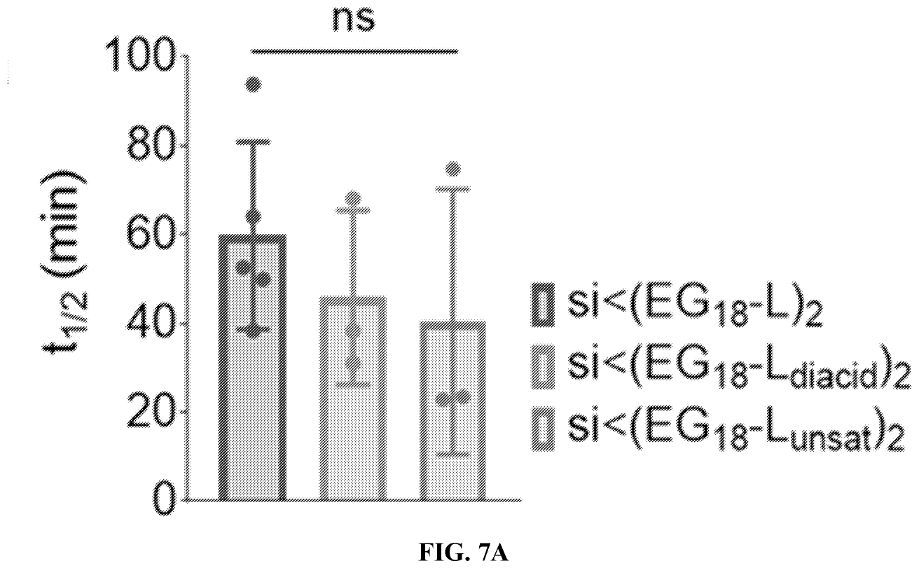

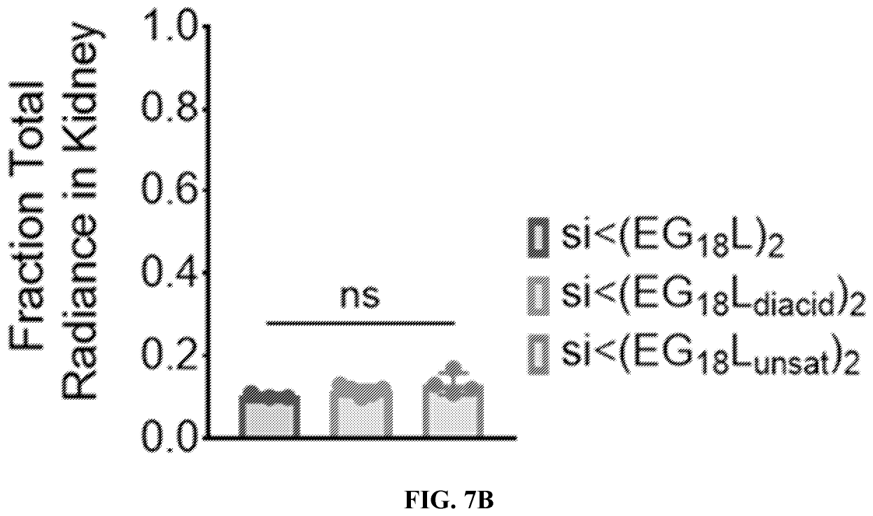

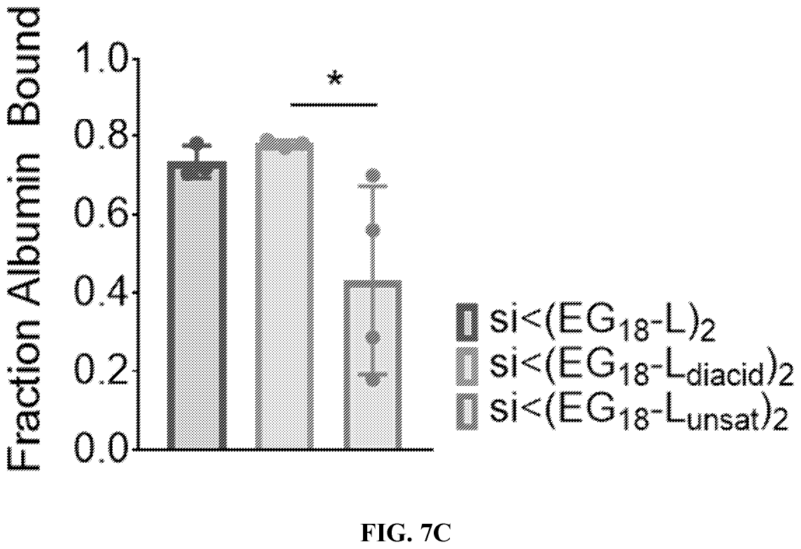

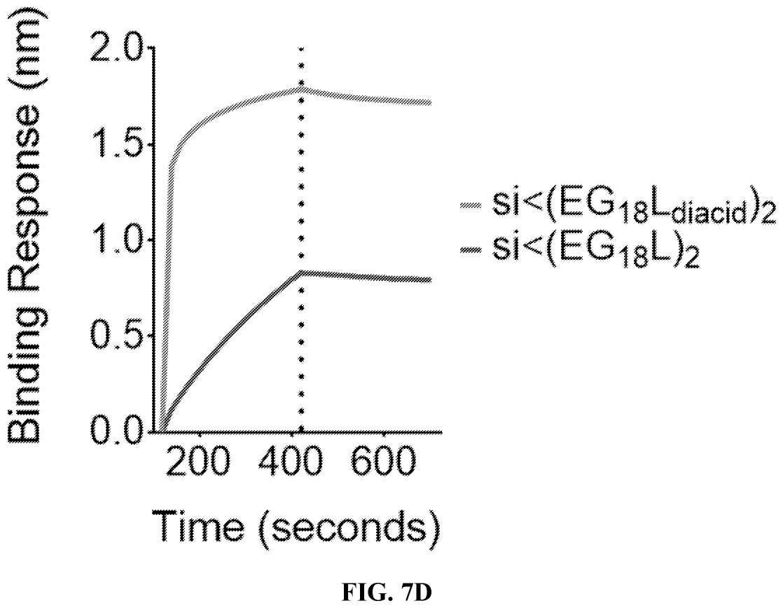

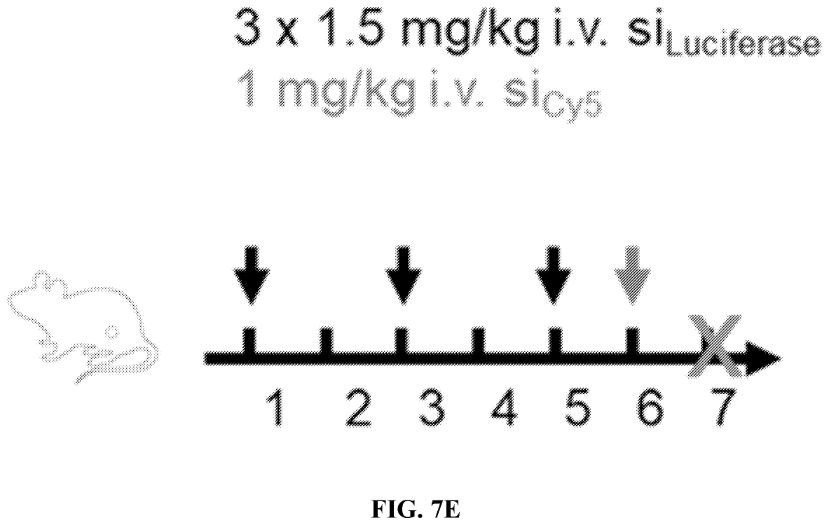

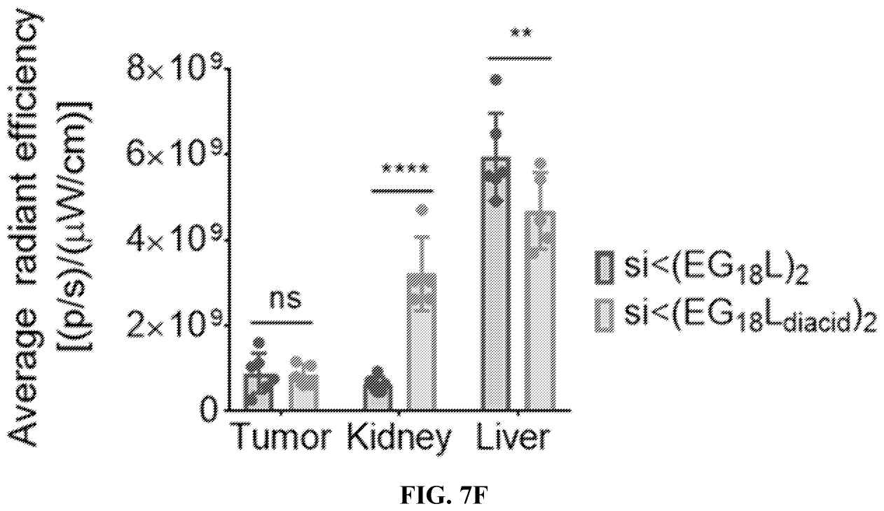

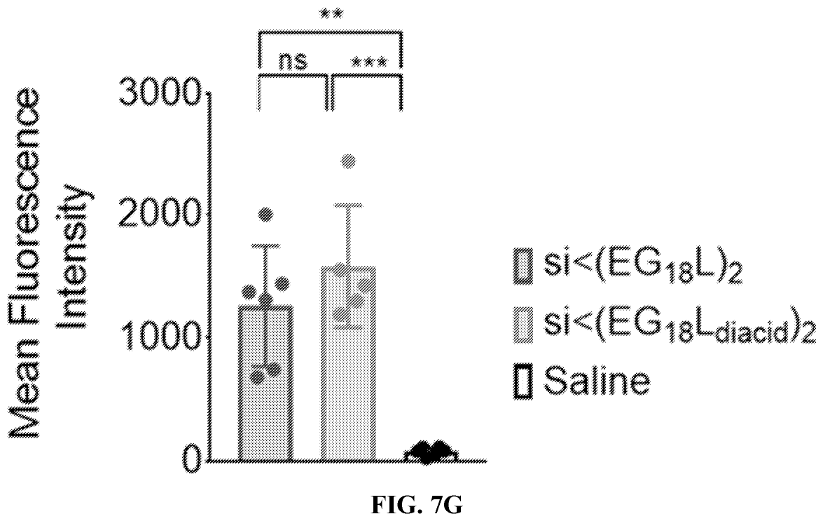

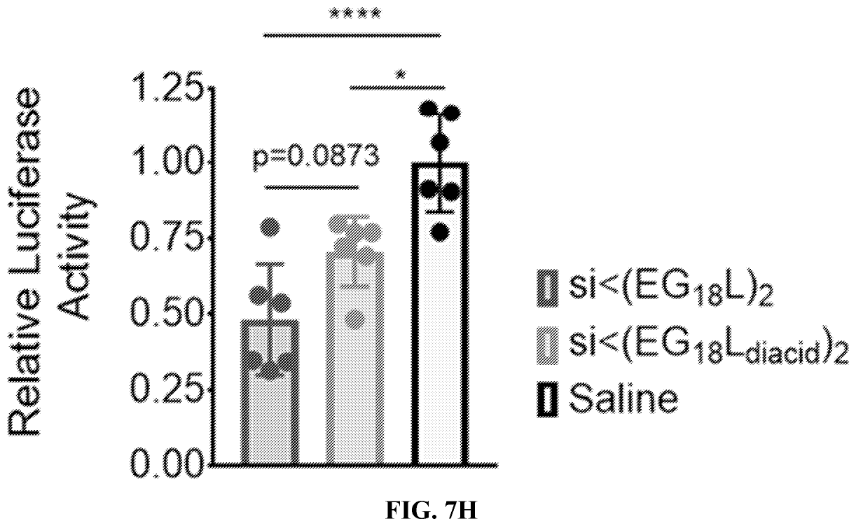

Further, interest in testing a variant with a double bond was motivated by 1) evidence suggesting that oleate, an unsaturated variant of stearate, possesses a higher affinity for albumin and 2) the idea that double bonds introduce “kinks” into lipid chains that prevent close packing and may therefore deter self-assembly, which was shown to correlate with poorer albumin binding. To test these hypotheses, si<(EG₁₈-Amine)₂ was synthesized and conjugated amine-reactive PFP-modified lipid variants on to the terminus of the conjugates. Both the diacid and unsaturated variants of the conjugate exhibited comparable kidney accumulation and absolute circulation half-life (FIG. 7A, FIG. 7B). However, si<(EG₁₈L_{unsaturated})₂ demonstrated significantly diminished albumin binding in vivo compared to its saturated and diacid counterparts (˜40% bound versus ˜75-80% bound) (FIG. 7C). Based on the correlations of albumin-bound in vivo and ultimate tumor gene silencing observed previously, only the diacid variant was further characterized. Strikingly, si<(EG₁₈L_{diacid})₂ showed a substantially stronger binding response to albumin (FIG. 7D), with affinity for human serum albumin approximately two orders of magnitude superior to its non-acid counterpart (si<(EG₁₈L_{diacid})₂ K_{D}=0.15±0.002 nM and si<(EG₁₈L)₂ K_{D}=30±0.3 nM). To determine whether the impact of this increased affinity for albumin would be captured by circulation half-life at longer time points, blood was sampled from mice injected intravenously with 5 mg/kg of either fluorescently labeled si<(EG₁₈L)₂ or its diacid counterpart but found no significant difference in their PK profiles.

Based on the comparable characteristics of the two variants to this point, the two were comparted in a tumor-bearing mouse model. The dose was decreased, compared to the previous screening, in an attempt to emphasize any differences between the variants (FIG. 7E). Conjugate targeting luciferase was administered intravenously every other day for a total of three injections of 1.5 mg/kg, with a non-functional, fluorescently labeled conjugate delivered 18 h prior to sacrifice. Tumor accumulation measured by epifluorescence and flow cytometry of cells demonstrated comparable accumulation of both versions of the conjugate (FIG. 7F, FIG. 7G). However, the diacid conjugate demonstrated significantly higher renal accumulation and lower liver accumulation. Despite the significant increase in albumin affinity of this conjugate, it did not produce significantly better reduction of luciferase activity. Indeed, si<(EG₁₈L)₂, trends toward having significantly more knockdown than its diacid counterpart (FIG. 711). Based on these data, it is hypothesized, without being bound by a particular theory, that hydrophobicity of the conjugate outcompetes the role of higher albumin affinity for effective in vivo silencing.

### Example 9

Albumin-Binding Lipophilic siRNA Conjugates Outperform siRNA Directly Conjugated to Albumin

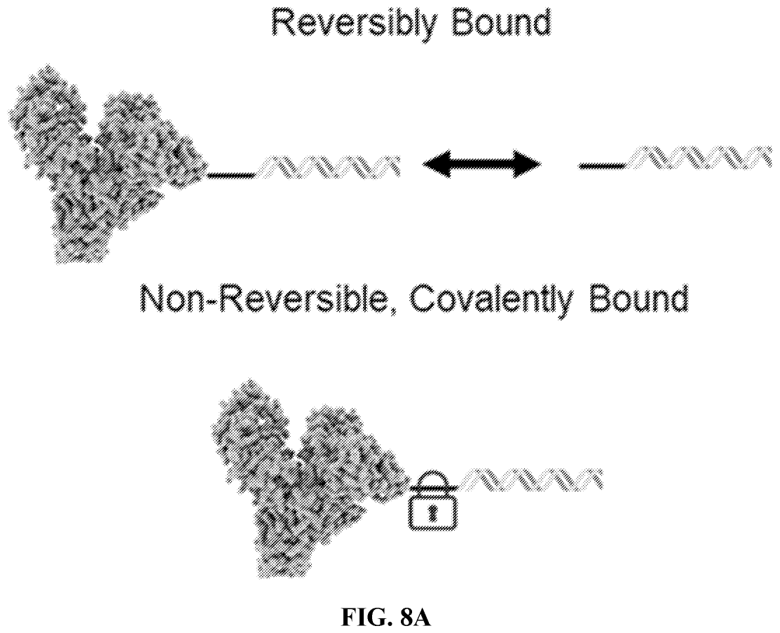

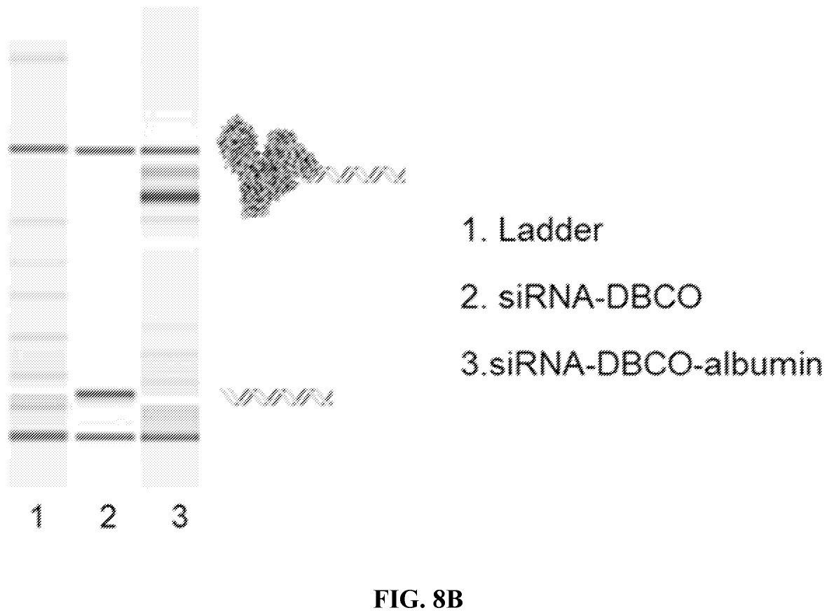

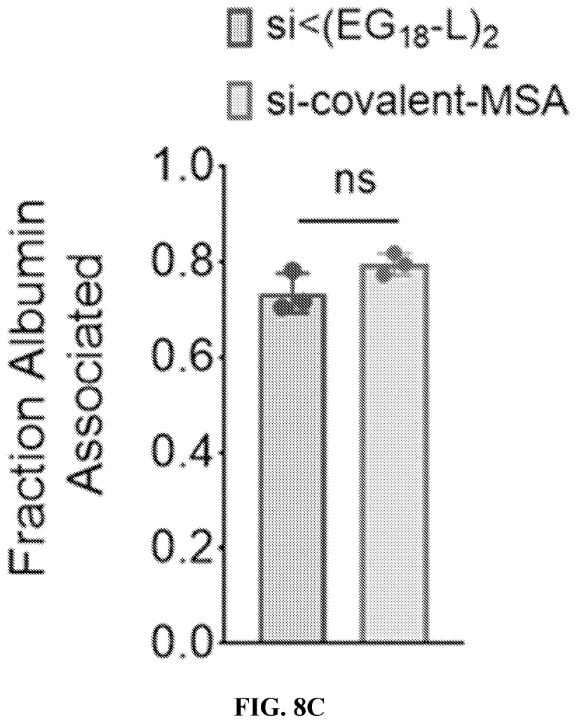

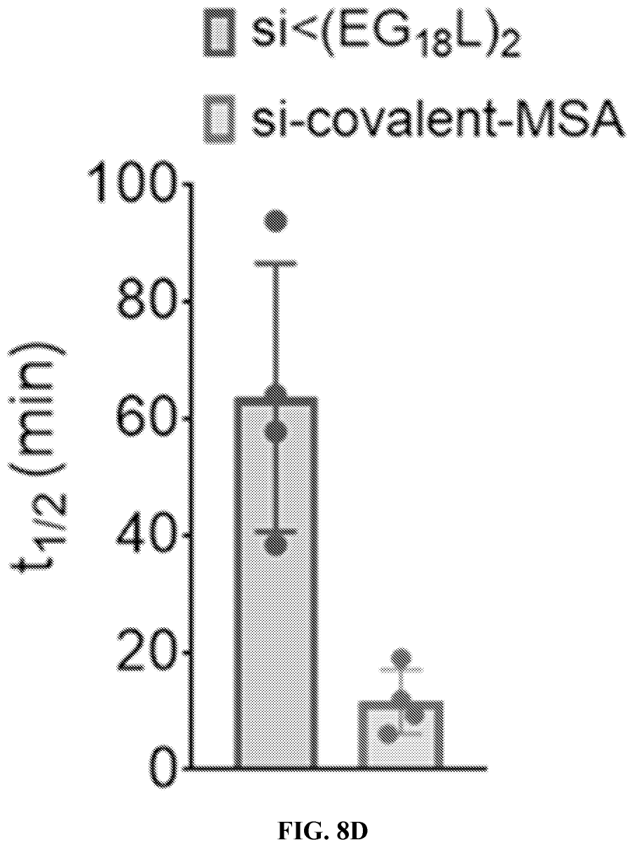

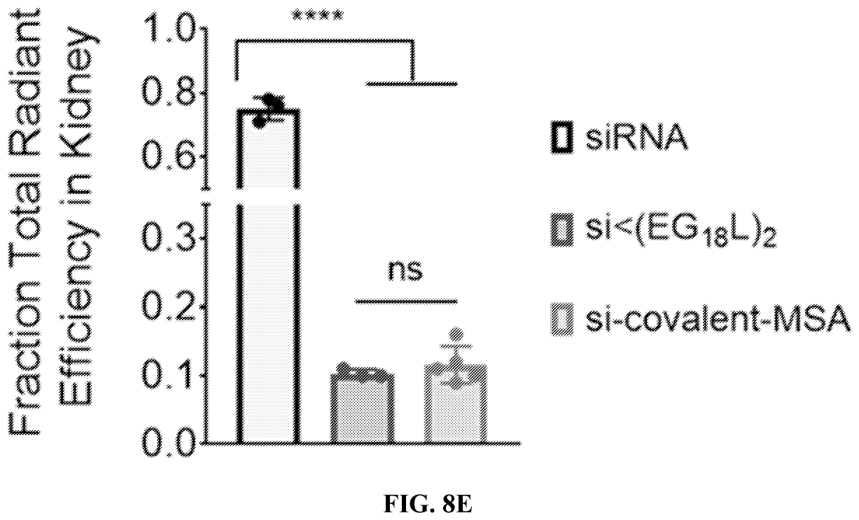

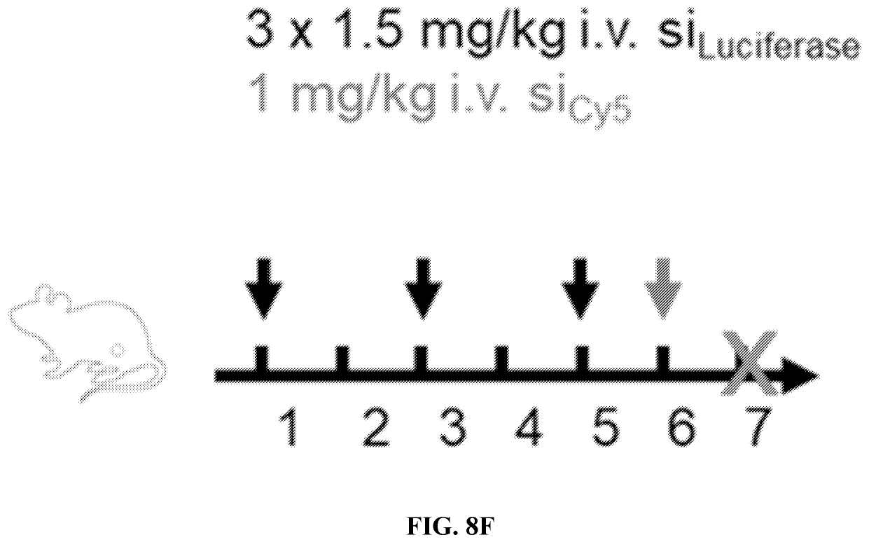

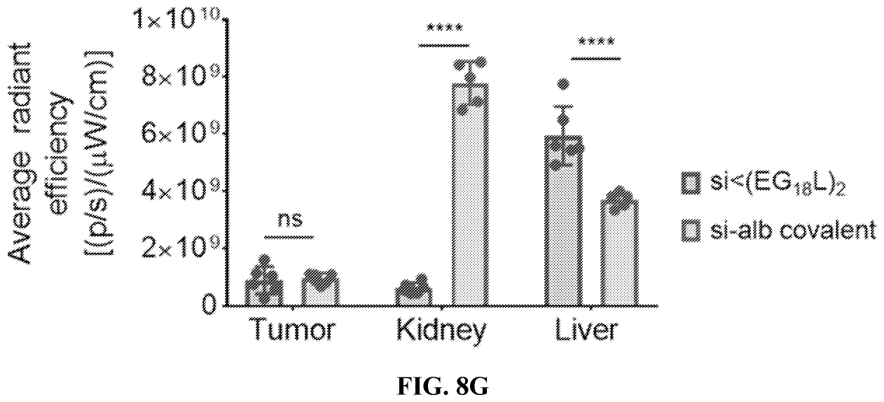

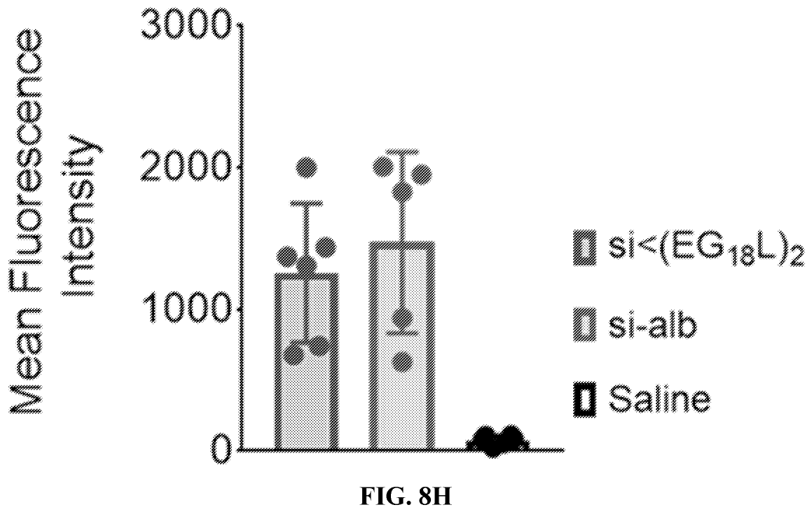

To further interrogate the appeal of an albumin-binding, lipophilic conjugate, an siRNA duplex was synthesized directly, covalently bound to mouse serum albumin. It was sought to determine whether the lipid-mediated, reversible binding was preferable to maximized albumin-bound delivery (FIG. 8A). These complexes were synthesized by leveraging the two free thiol groups present on mouse serum albumin as a handle for modifying with an azido-PEG₃-maleimide linker followed by reacting with DBCO-modified siRNA duplex. Gel electrophoresis demonstrated an upward shift of resulting product relative to the DBCO-duplex precursor, suggesting successful conjugation and removal of unreacted ligands (FIG. 8B). Fluorophore-labeled duplex was additionally used to validate that A260 readouts of product agreed with fluorescent readouts for quantification of siRNA in the resulting complex. Plasma isolated from mice injected with the siRNA-MSA complex demonstrated that approximately 80% of the siRNA was associated with fractions associated with albumin (FIG. 8C). Strikingly, however, the observed half-life of the siRNA covalently bound to albumin was greatly diminished compared to the lipophilic siRNA conjugate (FIG. 8D). Previous reports have shown that cell surface glycoproteins gp18 and gp30 can bind to covalently modified albumin and act as scavenger receptors that traffic the modified albumin for lysosomal degradation. This is further supported by the organ biodistribution of the siRNA taken from the same mice, which shows no significant difference in kidney levels between the groups (FIG. 8E). This suggests that liberation from the albumin resulting in renal clearance is not responsible for the reduction in circulation half-life. It was sought to compare the in vivo efficacy of the two using a rigorous, low dose regime to emphasize differences in performance between the two candidates (FIG. 8F). Mice bearing luciferase-expressing MDA-MB-231 tumors were treated intravenously every other day with 1.5 mg/kg siRNA for a total of three injections. A fluorescently labeled, inactive version of each compound was administered 18 h before sacrifice. Interestingly, epifluorescence of whole organs and flow cytometry of tumor cells demonstrates comparable tumor accumulation of the two compounds (FIG. 8G, FIG. 811). However, the conjugate demonstrates significantly better reduction of luciferase activity than its covalently bound counterpart (FIG. 8I). These findings suggest that non-covalent binding to albumin is preferable and, further, that the lipids on the conjugate may be important to escaping endosomes after uptake by tumor cells.

The examples disclosed herein show that systematic variation of lipid-siRNA conjugate valency, linker length, phosphorothioate bonds, lipid chemistry, and linker branching architecture impacts albumin binding, pharmacokinetics, tumor biodistribution, and carrier-free tumor gene silencing activity in vivo. It is shown that lipid valency has a significant impact on pharmacokinetics. Further, the addition of a hydrophilic linker improves albumin binding, but not indiscriminately. The data suggest that there is an advantageous length of hydrophilic linker, e.g., [si<(EG₁₈L)₂], and that this linker improves albumin-binding when placed after the branching point of the divalent moiety. Modest dosing with this construct achieved nearly 80% carrier-free knockdown in an orthotopic tumor model of triple negative breast cancer, a significant advance in the sector of the extrahepatic RNAi field developing carrier free siRNAs for cancer therapy. Notably, designs with the hydrophilic linker before the branching point of the divalent structure, when matched for both overall hydrophobicity or length between the siRNA and lipids, showed inferior albumin association in plasma in vivo, which may be attributable to greater lipoprotein association and propensity for self-assembly (lower CMC). The examples also showed that phosphorothioate, rather than phosphodiester, linkages are beneficial both within the linker structure and sense strand terminus where the albumin-binding moiety is located, possibly to promote plasma protein binding. It was additionally demonstrated that the increased albumin affinity conferred by keeping the carboxyl handle of the fatty acid intact is outcompeted by the need for hydrophobicity to achieve silencing efficacy. Further, the examples suggest that siRNA benefits from being reversibly, rather than covalently, bound to albumin.

Overall, this work has important implications for the delivery of siRNAs to extrahepatic targets, a goal that has remained clinically elusive. Albumin is known to accumulate at sites of inflammation and vascular leakiness. Therefore, the insights gleaned from the development of si<(EG₁₈L)₂ as a beneficial siRNA structure for in situ albumin piggybacking can have far reaching implications for the development and improvement of extrahepatic, carrier-free siRNA therapeutics in cancer and beyond.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A conjugate comprising: an oligonucleotide; a lipophilic ligand capable of binding albumin; and a linker attaching the oligonucleotide and the lipophilic ligand, the linker comprising a branching molecule attached to the oligonucleotide, and a hydrophilic spacer attaching the branching molecule and the lipophilic ligand.

Clause 2. The conjugate of clause 1, wherein the branching molecule includes at least one branch point having at least two independent branches.

Clause 3. The conjugate of clause 1 or 2, wherein the hydrophilic spacer comprises 1 to 100 hydrophilic blocks.

Clause 4. The conjugate of any one of clauses 1-3, wherein each hydrophilic block comprises 1 to 150 repeats of a hydrophilic compound.

Clause 5. The conjugate of clause 4, wherein the hydrophilic compound comprises ethylene glycol, zwitterionic linkers, peptoids, amino acids, poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), or a combination thereof.

Clause 6. The conjugate of clause 4, wherein each hydrophilic block comprises 1 to 100 repeats of ethylene glycol.

Clause 7. The conjugate of any one of clauses 1-6, wherein the hydrophilic blocks are attached to each other through phosphorothioate linkages.

Clause 8. The conjugate of any one of clauses 1-7, wherein the oligonucleotide comprises DNA, RNA, synthetic mimics of DNA or RNA, or a combination thereof.

Clause 9. The conjugate of any one of clauses 1-8, wherein the oligonucleotide comprises siRNA, miRNA or a single stranded antisense oligonucleotide.

Clause 10. The conjugate of any one of clauses 1-9, wherein the oligonucleotide comprises stabilizing modifications.

Clause 11. The conjugate of any one of clauses 1-10, wherein the oligonucleotide comprises a plurality of phosphorothioate linkages.

Clause 12. The conjugate of any one of clauses 1-11, wherein the oligonucleotide has about 15 nucleotides to about 40 nucleotides.

Clause 13. The conjugate of any one of clauses 1-12, wherein the lipophilic ligand comprises a lipid including a C₁₂-C₂₂ hydrocarbon chain.

Clause 14. The conjugate of any one of clauses 1-13, wherein the lipophilic ligand is divalent.

Clause 15. The conjugate of clause 14, wherein the lipophilic ligand comprises two independent lipids, each lipid including a C₁₂-C₂₂ hydrocarbon chain.

Clause 16. The conjugate of clause 15, wherein each lipid includes a C₁₈ hydrocarbon chain.

Clause 17. The conjugate of clause 13, wherein the lipid includes a carboxyl at its terminal end.

Clause 18. The conjugate of any one of clauses 2-17, wherein each branch is attached to an individual hydrophilic spacer, and each hydrophilic spacer is attached to an individual lipid of the lipophilic ligand.

Clause 19. The conjugate of any one of clauses 1-18, wherein the hydrophilic spacer is attached to the lipophilic ligand through a phosphorothioate linkage.

Clause 20. The conjugate of any one of clauses 1-19, wherein the conjugate has a binding affinity (K_{d}) to albumin of less than 1 μM.

Clause 21. The conjugate of any one of clauses 1-20 wherein the conjugate has a critical micelle concentration of greater than 1850 nM.

Clause 22. The conjugate of any one of clauses 1-21, wherein the conjugate comprises about 20% to about 60% phosphorothioate linkages based on a total amount of phosphate-based linkages in the conjugate.

Clause 23. A conjugate comprising: an siRNA; a lipophilic ligand capable of binding albumin, the lipophilic ligand comprising two independent lipids, each lipid including a C₁₈ hydrocarbon chain; and a linker attaching the siRNA and the lipophilic ligand, the linker comprising a branching molecule attached to the siRNA and including at least one branch point having at least two independent branches, and a hydrophilic spacer attaching an individual branch and an individual lipid, the hydrophilic spacer including 1 to 6 hydrophilic blocks, each hydrophilic block including 2 to 10 repeats of ethylene glycol.

Clause 24. A composition comprising: the conjugate of clause 1; and one or more pharmaceutically acceptable excipients.

Clause 25. The composition of clause 24, wherein the composition does not include a carrier composition.

Clause 26. A method of gene silencing, the method comprising administering the conjugate of clause 1 or the composition of clause 24 to a subject in need thereof.

Clause 27. The method of clause 26, wherein the conjugate is administered intravenously.

Clause 28. The method of clause 26 or 27, wherein the conjugate binds albumin following administration.

Clause 29. The method of any one of clauses 26-28, further comprising pre-complexing the conjugate with albumin prior to administering to the subject.

Clause 30. The method of any one of clauses 26-29, wherein the conjugate is administered without an associated carrier composition.

Sequences

| | Sequence (5′ to 3′) |
|---|---|
| Luciferase (Luc) | SEQ ID NO: 1-(PHO)(OMeU)*(fU)* |
| Antisense | (OMeC)(fA)(OMeU)(fU)(OMeA)(fU) |
| | (OMeC)(fA)(OMeG)(fU)(OMeG)(fC) |
| | (OMeA)(fA)(OMeU)*(fU)*(OMeG) |
| Luc Sense | SEQ ID NO: 2-(fC)*(OMeA)*(fA) |
| | (OMeU)(fU)(OMeG)(fC)(OMeA)(fC) |
| | (OMeU)(fG)(OMeA)(fU)(OMeA)(fA) |
| | (OMeU)(fG)*(OMeA)*(fA) |
| Scrambled (Scr) | SEQ ID NO: 3-(PHO)(OMeG)*(fU)* |
| Antisense | (OMeA)(fU)(OMeU)(fA)(OMEU)(fA) |
| | (OMeC)(fG)(OMeC)(fG)(OMeA)(fU) |
| | (OMeU)(fA)(OMeA)(fC)(OMeG)*(fA)* |
| | (OMeC) |
| Scr Sense | SEQ ID NO: 4-(fC)*(OMeG)*(fU) |
| | (OMeU)(fA)(OMeA)(fU)(OMeC)(fG) |
| | (OMeC)(fG)(OMeU)(fA)(OMEU)(fA) |
| | (OMeA)(fU)*(OMeA)*(fC) |
| Cy5-labeled Luc | SEQ ID NO: 5-(Cy5)(OMeU)*(fU)* |
| Antisense | (OMeC)(fA)(OMeU)(fU)(OMeA)(fU) |
| | (OMeC)(fA)(OMeG)(fU)(OMeG)(fC) |
| | (OMeA)(fA)(OMeU)*(fU)*(OMeG) |
| Biotin-TEG- | SEQ ID NO: 6-(BTN-TEG)*(OMeU)* |
| labeled Luc | (fU)*(OMeC)(fA)(OMeU)(fU)(OMeA) |
| Antisense | (fU)(OMeC)(fA)(OMeG)(fU)(OMeG) |
| | (fC)(OMeA)(fA)(OMeU)*(fU)*(OMeG) |
| Mcl-1 Antisense | SEQ ID NO: 7- |
| | (fC)*(OMeA)*(fU)(OMeC)(fG)(OMeA) |
| | (fA)(OMeC)(fC)(OMeA)(fU)(OMeU) |
| | (fA)(OMeG)(fC)(OMeA)(fG)*(OMeA)* |
| | (fA) |
| Mcl-1 Sense | SEQ ID NO: 8-(PHO)(OMeU)*(fU)* |
| | (OMeC)(fU)(OMeG)(fC)(OMeU)(fA) |
| | (OMeA)(fU)(OMeG)(fG)(OMeU)(fU) |
| | (OMeC)(fG)(OMeA)*(fU)*(OMeG) |

Phosphorothioate bond (X)*(X)
Phosphodiester bond (X)(X)
2′F substituted base (fX)
2′OMe substituted base (OMeX)

1. A conjugate comprising:
an oligonucleotide;
a lipophilic ligand capable of binding albumin; and
a linker attaching the oligonucleotide and the lipophilic ligand, the linker comprising
a branching molecule attached to the oligonucleotide, and
a hydrophilic spacer attaching the branching molecule and the lipophilic ligand.

2. The conjugate of claim 1, wherein the branching molecule includes at least one branch point having at least two independent branches.

3. The conjugate of claim 1, wherein the hydrophilic spacer comprises 1 to 100 hydrophilic blocks.

4. The conjugate of claim 3, wherein each hydrophilic block comprises 1 to 150 repeats of a hydrophilic compound.

5. The conjugate of claim 4, wherein the hydrophilic compound comprises ethylene glycol, zwitterionic linkers, peptoids, amino acids, poly(glycerols), poly(oxazoline), poly(acrylamide), poly(N-acryloyl morpholine, poly(N,N-dimethyl acrylamide), poly(2-hydroxypropyl methacrylamide), poly(2-hydroxyethyl methacryalmide), or a combination thereof.

6. The conjugate of claim 4, wherein each hydrophilic block comprises 1 to 100 repeats of ethylene glycol.

7. The conjugate of claim 4, wherein the hydrophilic blocks are attached to each other through phosphorothioate linkages.

8. The conjugate of claim 1, wherein the oligonucleotide comprises DNA, RNA, synthetic mimics of DNA or RNA, or a combination thereof.

9. The conjugate of claim 1, wherein the oligonucleotide comprises siRNA, miRNA or a single stranded antisense oligonucleotide.

10. (canceled)

11. The conjugate of claim 1, wherein the oligonucleotide comprises a plurality of phosphorothioate linkages.

12. The conjugate of claim 1, wherein the oligonucleotide has about 15 nucleotides to about 40 nucleotides.

13. The conjugate of claim 1, wherein the lipophilic ligand comprises a lipid including a C₁₂-C₂₂ hydrocarbon chain.

14. The conjugate of claim 1, wherein the lipophilic ligand is divalent.

15. The conjugate of claim 1, wherein the lipophilic ligand comprises two independent lipids, each lipid including a C₁₂-C₂₂ hydrocarbon chain.

16. The conjugate of claim 15, wherein each lipid includes a C₁₈ hydrocarbon chain.

17. (canceled)

18. The conjugate of claim 2, wherein each branch is attached to an individual hydrophilic spacer, and each hydrophilic spacer is attached to an individual lipid of the lipophilic ligand.

19. The conjugate of claim 1, wherein the hydrophilic spacer is attached to the lipophilic ligand through a phosphorothioate linkage.

20. The conjugate of claim 1, wherein the conjugate has a binding affinity (K_{d}) to albumin of less than 1 μM.

21-23. (canceled)

24. A composition comprising:
the conjugate of claim 1; and
one or more pharmaceutically acceptable excipients.

25. (canceled)

26. A method of gene silencing, the method comprising administering the conjugate of claim 1 to a subject in need thereof.

27-30. (canceled)
